SEQUENCE LISTING (5 sequences)

SEQ ID NO: 1 - DNA, 1008 bp, Mus sp.
tggagctcagaggcagagcacttgtctagcataaacaaccctgggttaatccgagctccaacagggaaacagtctgcagactgagagaaccgagcattctatcagaaccccgcagctctggattctaggtccagcagcaaccagagagaccatgcaggtcatccttgctgtgatagtctggatggcttcgtctgtgtctagttcttcatttacagcaagcactttgccagatataacaaacgaggacttcattaaagaatgtgttcaagttcacaaccagcttcggtcaaaagtgagtccaccagcccggaatatgctgtacatgtcttgggacccaaaactagcccaaattgcaaaagcatggacaaaatcttgtgaatttaaacacaacccacagctgcattcacggatacacccaaatttcaccgccctgggagagaatatctggcttggctctctatccatcttttcagtatcctcagccatctctgcctggtatgaagaaattaagcactatgacttcagcactaggaaatgtagacatgtctgtggccattatactcaggttgtttgggcagacagttacaaacttggctgtgcagtgcaactttgccctaatggagcaaattttatatgcgactatggaccagcaggaaattacccaacgtggccatataagcaaggagccacgtgcagtgattgcccaaaagatgacaagtgtctcaacagtctctgattaacccacgacgagaccaggtctcacgttactactctgtcgcattatccagactggcctatatacctgcgtaacagatacacatctctctttctcattgctaagtcggttctcctattactgtctgttataattaccatctgggtaaagcacaaatatcctaacttggttcttttggactaaagctgtggttgggggacaactgaatcacatgcggctatttaaaaacttttcaataaaatctcagtcaaaagaaaaaaaaaaaaaaaaaa

SEQ ID NO: 2 - protein, 255 aa, Mus sp.
MQVILAVIVWMASSVSSSSFTASTLPDITNEDFIKECVQVHNQLRSKVSPPARNMLYMSWDPKLAQIAKAWTKSCEFKHNPQLHSRIHPNFTALGENIWLGSLSIFSVSSAISAWYEEIKHYDFSTRKCRHVCGHYTQVVWADSYKLGCAVQLCPNGANFICDYGPAGNYPTWPYKQGATCSDCPKDDKCLNSLCINPRRDQVSRYYSVDYPDWPIYLRNRYTSLFLIAKSVLLLLSVIITIWVKHKYPNLVLLD

SEQ ID NO: 3 - protein, 266 aa, Homo sapiens
MRVTLATIAWNVSFVSNYSHTANILPDIENEDFIKDCVRIHNKFRSEVKPTASDMLYMIWDPALAQIAKAWASNCQFSHNTRLKPPHKIHPNFTSLGENIWTGSVPIFSVSSAITNWYDEIQDYDFKTRICKKVCGHYTEVVWADSYKVGCAVQFCPKVSGFDALSNGAHFICNYGPGGNYPTWPYKRGATCSACPNNDKCLDNLCVNRQRDQVKRYYSVVYPGWPIYPRNRYTSLFLIVNSVILILSVIITILVQLKYPNLVLLD

SEQ ID NO: 4 - protein, 42 aa, Mus sp.
  MISC_FEATURE (11)..(32): some may be missing
RRRCWWGYYYXXXXXXXXXXXXXXXXXXXXXXRRRCWWGYYY

SEQ ID NO: 5 - protein, 9 aa, Homo sapiens
KVSGFDALS

(19) United States
(12) Patent Application Publication
(10) Pub. No.: US 2005/0187153 A1
(43) Pub. Date: Aug. 25, 2005

(54) RTVP based compositions and methods for the treatment of prostate cancer, autoimmunity and infectious disease

(75) Inventor: Timothy Charles Thompson, Houston, TX (US)

Correspondence Address: VINSON & ELKINS L.L.P., 1001 FANNIN STREET, 2300 FIRST CITY TOWER, HOUSTON, TX, 77002-6760, US

(73) Assignee: Baylor College of Medicine, Houston, TX (US)

(21) Appl. No.: 10/909,864

(22) Filed: Jul. 30, 2004

Related U.S. Application Data
(63) Continuation of application No. 10/417,727, filed on Apr. 17, 2003.
(63) Continuation of application No. 09/876,225, filed on Jun. 8, 2001, now abandoned.
(60) Provisional application No. 60/491,858, filed on Aug. 1, 2003.

(51) Int. Cl. A61K 38/17

(52) U.S. Cl. 514/12

(57) ABSTRACT

This invention relates to a gene encoding RTVP that has been shown to be up-regulated by p53 using differential display-PCR and subsequently by co-transfection studies. RTVP-1 mRNA is abundant in normal mouse and human prostatic epithelial cells and primary tumors, but is significantly down regulated in metastatic mouse and human prostate cancer. In prostate cancer cells overexpression of the mouse RTVP-1 gene (mRTVP-1) induced apoptosis that was accompanied by increased caspase 8, 9 and 3 activities. mRTVP-1-stimulated apoptosis was also associated with increased levels of bax, bad and activated BID; reduced levels of bcl-2 and bcl-X_{L}; and cytosolic cytochrome c accumulation. Adenoviral-vector-mediated mRTVP-1 expression lead to potent growth suppression and antimetastatic activities in an orthotopic mouse model of prostate cancer in vivo. These therapeutic activities were associated with anti-angiogenic effects and importantly a local and systemic immune response. Accordingly, p53 was linked with suppression of metastasis through its induction of mRTVP-1, which can concurrently induce apoptosis, suppress angiogenesis and stimulate an antitumor immune response. Furthermore, studies of RTVP negative mice have shown that RTVP may be potent effecter of the immune system indicating that manipulation of RTVP expression or fragments thereof can provide effective agents for the prevention and treatment of autoimmune disorders and infectious diseases. Thus, the invention includes compositions and methods, based on RTVP nucleic acid, polypeptides, and antibodies, for use in the treatment, prevention and detection of neoplastic disease (specifically, metastatic prostatic neoplasia), autoimmune disorders and infectious diseases.

### RELATED APPLICATIONS

The present application claims priority to U.S. Provisional Application No. 60/491,858 filed Aug. 1, 2003 and U.S. patent application Ser. No. 09/876,225 filed Jun. 8, 2001.

### RIGHTS IN THE INVENTION

This invention was made, in part, with United States government support under grant number R01-50588, awarded by the National Cancer Institute, and also grant number P50-58204, awarded by the National Cancer Institute, Specialized Program or Research Excellence (SPORE), and the United States Government has certain rights in the invention.

### BACKGROUND OF THE INVENTION

1. Field of the Invention

The present invention relates to RTVP genes and associated sequences, to RTVP proteins, and to methods and tools using these sequences for the diagnosis, study and treatment of disease. Further, the invention includes RTVP receptor protein and the gene which encodes this protein. In particular, the invention relates to compositions and methods based on RTVP for the treatment, prevention and detection of prostatic neoplasia (such as prostate carcinoma and associated metastatic disease), autoimmunity, immune suppression and infectious diseases.

2. Description of the Background

The prostate is a walnut size gland that is part of the male reproductive system that makes and stores seminal fluid. It is located below the bladder, in front of the rectum, and surrounds the upper part of the urethra. To work properly, the prostate needs male hormones androgens). The main male hormone is testosterone, which is produced by the testicles. Prostate cancer or PC is characterized by the uncontrolled growth of prostate epithelial cells to form one or more tumors. Localized PC is treated very successfully and results in a 100% five-year survival rate. Metastatic PC has a 31% survival rate, about 80% of which attacks the bone with a significant portion of the remainder attacking the lungs. PC that metastasizes to the bone is not bone cancer and is treated as later stage or distant PC.

The American Cancer Society reports that excluding skin cancer, PC is the most common malignancy and the second leading cause of cancer death among men in the US. The incidence and mortality of PC increase with age, 77% of men with new diagnoses of prostate cancer each year are over the age of 65. PC is rare in younger men, with an incidence rate of less than one case per 100,000 for men under age 40. However, the rate climbs to 82 per 100,000 for men ages 50-54, 518 for ages 60-64, and 1,326 for ages 70-74. African-Americans are twice as likely to develop and die from PC than men of other ethnic and racial groups.

The number of PC cases will increase dramatically during the next four decades as the demographics of the Baby Boom generation take effect and the reduction of deaths from cardiovascular and smoking-related diseases increase the size of the 60-84 year old population segment. It is estimated that approximately 570,000 new cases of PC will be diagnosed in 2030 (when there will be close to 50 million men in this population group), and estimated over 2,000,000 men in treatment. This compares to an incidence of 168,665 new cases in 1995.

Surgery and/or radiotherapy remain the treatments of choice for early PC. Typically, surgery requires complete removal of the prostate (radical prostatectomy), and quite often removal of surrounding lymph nodes (lymphadenectomy). Radiotherapy, which is also used as adjuvant therapy, may be either external or interstitial and involves exposure of the effected tissue to radioisotopes such as ¹²⁵I. With more advanced forms of PC, endocrine therapy is often the preferred treatment. The aim of this therapy is to deprive prostate cells, and presumably transformed prostate cells as well, of testosterone. This can be accomplished by orchiectomy (castration), or administration of drugs (e.g. leuprolide, goserelin), antiandrogens (e.g. flutamide and bicalutamide), estrogens or synthetic hormones that are agonists of luteinizing hormone-releasing hormone, which directly inhibit testicular and organ synthesis and suppress luteinizing hormone secretion which in turn leads to reduced testosterone secretion by the testes. Despite the advances made in achieving a pharmacologic orchiectomy, the survival rates for those with late stage carcinomas are poor.

In its more aggressive form, transformed prostatic tissues escape from the prostate capsule and metastasize invading locally and throughout the bloodstream and lymphatic system. Metastasis, defined as tumor implants which are discontinuous with the primary tumor, can occur through direct seeding, lymphatic spread and hematogenous spread. All three routes have been found to occur with PC. Local invasions typically involve the seminal vesicles, the base of the urinary bladder, and the urethra. Direct seeding occurs when a malignant neoplasm penetrates a natural open field such as the peritoneal, pleural or pericardial cavities. Cells seed along the surfaces of various organs and tissues within the cavity or can simply fill the cavity spaces. Hematogenous spread is typical of sarcomas and carcinomas. Hematogenous spread of prostatic carcinoma occurs primarily to the bones, but can include massive visceral invasion as well. It has been estimated that a majority of newly diagnosed prostate cancer patients will have metastases at the time of initial diagnosis.

Many studies have shown that there exists a specific metastasis suppressor role for p53, a well-known tumor suppressor protein, in PC (reviewed in⁵). Initial experimental results using an in vivo mouse model of PC metastasis demonstrated that loss of p53 function can lead to the development of metastases that seed from relatively small numbers of cells within the primary tumor⁶. Subsequent studies demonstrated that although p53 mutations in human primary PC tissues are heterogeneous and relatively infrequent, they occur at significant levels in metastatic disease, ranging from 21%-30% mutation frequency in lymph node metastasis to higher than 90% mutation frequency in androgen-insensitive disseminated disease⁷⁻¹² This pattern of mutations suggests that only a few cells harboring p53 mutation in the primary tumor can seed metastases that clonally expand at distant sites. Consequently, there is a need for improved research tools, diagnostic tools and therapies, useful for the diagnosis, treatment and prevention of PC and metastasis associated with transformed prostate cells.

In addition, the treatment and prevention of various immune disorders, ranging from autoimmunity to graft rejection, could greatly benefit from the discovery and use of alternative methods or compositions in the suppression of the immune system. Compositions which are capable of inhibiting immune stimulatory effectors are likely candidates for use in such methods or compositions. While, on the other hand, the administration of methods or compositions which are capable of inducing or increasing an immune response would find multiple uses in the treatment and prevention of infectious diseases.

### SUMMARY OF THE INVENTION

The present invention overcomes the problems and disadvantages associated with current strategies and designs and provides new diagnostic, therapeutic and research tools, arid methods based on certain proteins related to testes-specific, vespid and pathogenic proteins (RTVP). The invention also relates to compositions and kits comprising the RTVP gene, the RTVP polypeptide and active fragments thereof, and antibodies thereto.

One embodiment of the invention is directed to isolated nucleic acids comprising the sequence of the RTVP gene. The gene may comprise the entire sequence of RTVP, all or part of the sequence. Further, the invention includes the RTVP polypeptide encoded therein, and active portions thereof. Genes may be functionally linked to vectors such as plasmid or viral vectors and he capable of expression in a suitable host.

Another embodiment of the invention is directed to recombinant cells containing nucleic acid segments that encodes RTVP polypeptides and portions thereof. Cells may be eukaryotic or prokaryotic, and may express the protein or act simply as biological containers of the gene sequence.

Another embodiment of the invention is directed to antibodies reactive against the RTVP protein and to antibodies that maybe reactive to antigenically active portions thereof. Antibodies maybe polyclonal or monoclonal, recombinant or synthetic. A further embodiment of the invention is directed to hybridomas that express antibodies to the RTVP polypeptide or to antigenic portions thereof.

Another embodiment of the invention is directed to the receptor of RTVP protein, and to compositions and methods useful for the treatment of neoplastic diseases such as prostate cancer and related metastasis. A further embodiment is directed to the gene for the receptor protein as well as to associated promoter and other transcription or translation controlling sequences. A further embodiment includes antibodies to the RTVP receptor protein which may also be useful for the treatment of neoplastic diseases such as prostate cancer and metastasis.

Another embodiment of the invention is directed to kits for the detection of prostatic disease. Kits comprise all or characteristic portions of the RTVP gene or the RTVP polypeptide sequence, or to antibodies to either the polypeptide or nucleic acid sequences, and are useful for detection of disease.

Another embodiment of the invention is directed to nucleic acid sequences that comprise the anti-sense of the RTVP gene or representative portions thereof. Sequences may be useful in compositions to for the treatment of prostatic disease by reducing or shutting down RTVP expression in cells.

Another embodiment of the invention is directed to compositions comprising RTVP or RTVP receptor polypeptides, or active portions thereof, as pharmaceutical compositions. Compositions may be useful in therapy, prophylaxis, diagnosis, or as research tools, and may further comprise pharmaceutically acceptable carriers for use in the treatment or prevention of diseases such as prostate cancer and metastatic disease.

Another embodiment of the invention is directed to the receptor of RTVP protein, and to compositions and methods useful for the treatment of autoimmune disorders and infectious diseases. A further embodiment includes antibodies to the RTVP receptor protein which may also be useful for the treatment of autoimmune disorders and infectious diseases.

Another embodiment of the invention is directed to kits for the detection of autoimmune disorders and infectious diseases. Kits comprise all or characteristic portions of the RTVP gene or the RTVP polypeptide sequence, or to antibodies to either the polypeptide or nucleic acid sequences, and are useful for detection of autoimmune disorders and infectious diseases.

Another embodiment of the invention is directed to nucleic acid sequences that comprise the anti-sense of the RTVP gene or representative portions thereof, wherein the sequences may be useful in compositions to for the treatment of autoimmune disorders by reducing or shutting down RTVP expression in cells.

Another embodiment of the invention is directed to compositions comprising RTVP or RTVP receptor polypeptides, or active portions thereof, as pharmaceutical compositions, wherein such compositions may be useful in therapy, prophylaxis, diagnosis, or as research tools, and may further comprise pharmaceutically acceptable carriers for use in the treatment or prevention of autoimmune disorders and infectious diseases.

Another embodiment of the invention is directed to methods for stimulating the immune system such as, for example, cytokines and growth factors, by administering composition of the invention to patients. Compositions may be administered in a therapeutically safe and effective dose to humans and other mammals in the form of pills, tablets, powder, liquid or combinations thereof. Furthermore, such embodiments may include compositions comprising RTVP or RTVP receptor polypeptides, or active portions thereof, as pharmaceutical compositions having therapeutic effects on individuals suffering from infectious diseases.

Other embodiments and advantages of the invention are set forth in part in the description which follows, and in part, will be obvious from this description, or may be learned from the practice of the invention.

### DESCRIPTION OF THE FIGURES

FIG. 1A (a) Nucleic acid sequence of mouse RTVP-1 (SEQ ID NO: 1); and
(b) comparison of mouse (SEQ ID NO. 2) with human (SEQ ID NO. 3) RTVP-1 amino acid sequence.

FIG. 2 Identification of RTVP-1 regulation by p53.

FIG. 3 In situ hybridization of human prostate cancer.

FIG. 4 mRTVP-1 induction of apoptosis through mitochondrial death pathway.

FIG. 5 RTVP-1 suppression of tumor growth and metastasis.

FIG. 6 Local and systemic immune response in AdmRTVP-1 treated tumors.

FIG. 7 (a) Molecular pathways in prostate cancer, (b) bystander effect of p53 gene therapy, and (c) p53 regulation of prostate cancer.

FIG. 8 Wild-type p53 regulation of mRTVP-1 promoter constructs.

FIG. 9 Induction of mRTVP-1.

FIG. 10 RTVP-1 expression in mouse and human tissue.

FIG. 11 Induction of apoptosis by mRTVP-1.

FIG. 12 Activation of mitochondrial pathways by mRTVP-1.

FIG. 13 Caspase activation by mRTVP-1.

FIG. 14 Regulation of apoptosis in prostate cancer.

FIG. 15 Induction of apoptosis and morphological changes by AdmRTVP-1.

FIG. 16 Increased survival of mice with orthotopic tumors after AdmRTVP-1 treatment.

FIG. 17 Apoptosis in 178-2BMA orthotopic tumors.

FIG. 18 (a) Immune cell infiltration in 178-2BMA orthotopic tumors, and (b) immune response in animals with 178-2BMA orthotopic tumors.

FIG. 19 Cytokine induction by mRTVP-1.

FIG. 20 Activation of JNK pathway by mRTVP-1.

FIG. 21 Schematic showing potential mechanisms of mRTVP-1-mediated suppression of metastasis.

FIG. 22 Demethylation of 148-1PA cells induces RTVP-1 expression.

FIG. 23 Schematic showing the targeted disruption of the RTVP-1 gene in a construct used to generate knock-out mice.

FIG. 24 Analysis of the effect of RTVP-1 on the stimulation of dendritic cells as measured through the activation marker CD80.

FIG. 25 Analysis of the effect of RTVP-1 on the stimulation of dendritic cells as measured through the activation marker CD80.

FIG. 26 Analysis of the effect of RTVP-1 on the stimulation of dendritic cells as measured through MCH II expression.

FIG. 27 Analysis of the effect of RTVP-1 on humoral effecter cells.

FIG. 28 Loss of RTVP-1 expression decreases allogenic CTL responses.

FIG. 29 RTVP-1 mechanism of action: tumor suppressor activity.

FIG. 30 Dendritic cells from RTVP-1+/+ and RTVP-1−/− mice have similar antigen uptake.

FIG. 31 RTVP-1−/− mice have more multinucleated giant cells in the spleen.

FIG. 32 Detection of immune cells in lymph nodes by immunohistochemistry.

FIG. 33 Higher cell turnover in RTVP−/− lymphoid organs.

### DESCRIPTION OF THE INVENTION

As embodied and broadly described herein, the present invention is directed to nucleic acid sequences encoding RTVP or the RTVP receptor protein, or active portions thereof, to RTVP and RTVP receptor polypeptides and functional portions thereof, to antibodies to thereto, and to compositions, kits and methods based on RTVP for the treatment, prevention and detection of disease, and specifically prostate disease, autoimmune disorders and infectious diseases, in mammals.

It has been shown that there exists a specific and established association between loss of p53 function and prostate cancer metastasis. Recent studies demonstrated that specific p53 mutations are clonally expanded in metastatic prostate cancer¹¹ and that a pattern of aberrant p53 expression in primary tumors, termed “clustered p53 staining,” has significant prognostic value in predicting recurrence following radical prostatectomy.^{12,13} It is generally considered that the nature of functional alterations which occur in cells containing p53 mutations specifically facilitates metastatic seeding, survival, and growth at distant metastatic sites. These alterations likely result, in part, from aberrant regulation of genes under the transcriptional control of p53 that have previously been shown to mediate apoptosis^{1, 14-17} and anti-angiogenic activities.¹⁸⁻²²

Surprisingly, it has been discovered that certain proteins related to testes-specific, vespid and pathogenic proteins (RTVP), and specifically mouse RTVP-1, are up-regulated by p53 in mouse prostate cancer cells. Proteins which are Related to Testes-specific, Vespid and Pathogenic proteins include those protein with homology to mammalian testes-specific proteins (e.g. TPXI), plant pathogenesis-related proteins (e.g. PR-protein such as subtype 1, PR-1b), vespid venom allergan proteins (antigen 5), or combinations thereof. Homology means that there is a relevant degree of similarity in the amino acid sequence between a polypeptide of the invention and one or more of the proteins mammalian testes-specific proteins, plant pathogenesis proteins and vespid venom allergan antigen 5, or in the respective gene sequences. Relevant homology means that the degree of amino acid sequence similarity is about 35% or greater, preferably about 45% or greater, more preferably about 60% or greater, and even more preferably about 75% or greater. Homology can be determined directly by sequencing the polypeptide of interest and comparing it with the known sequence, or experimentally by methods well know to those or ordinary skill in the art. Homology can be determined using, for example, blastp queries at default settings for amino acid homology determinations, and using blastn queries at default settings for nucleic acid homology determinations. Although the human RTVP gene was previously isolated^{23,24}, the functional significance of RTVP expression in any biological context has not previously been reported. RTVP-1 has also been shown to have immunostimulatory abilities making it useful in the treatment and prevention of infectious diseases. Further, human RTVP is significantly homologous (about 50% or greater) to mouse RTVP-1 (Table I). Loss of RTVP-1 expression is associated with mouse and human prostate cancer metastasis. Importantly, it has also been discovered that rnRTVP-1 has pro-apoptotic, anti-angiogenic, and immunostimulatory activities. This multifaceted role for mRTVP-1 in suppression of prostate cancer metastasis has also been shown using adenoviral-vector-mediated mRTVP-1 expression to suppress growth and metastasis of prostate cancer in vivo. Further, based on the presence of an extracellular protruding domain of the RTVP protein and the results of media-transfer experiments, therapeutic control of disease may also be achieved by targeting an RTVP receptor protein.

Accordingly, the present invention is directed to a novel gene, RTVP, which is under the regulation of p53 in human and mouse prostate cancer cells and potentially other normal and malignant cells. The sequence was identified using cell lines derived from mouse prostate cancer generated by the mouse prostate reconstitution model system. Briefly, a primary mouse prostate cancer cell line (148-1PA) was infected with an adenoviral vector containing wild-type human p53 and with a control virus without any added exogenous genes. The p53-induced and control mRNAs were used to generate cDNAs, and a fragment was isolated using differential display-PCR (DD-PCR) techniques. This fragment encoded the novel mouse protein, RTVP-1. Upon sequencing the DD-PCR fragment, it was determined that this mouse cDNA may be related to the human mRNA for RTVP-1 protein/human glioma pathogenesis-related protein, as there appeared to be significant homology at the nucleotide level.

Full-length cDNA for RTVP-1 was cloned using a cDNA library prepared from 148-1PA cells, its expression pattern in normal mouse prostate cancer tissues was further analyzed. Expression for this protein at the mRNA level appeared to be relatively low in most tissues examined including prostate, yet mRNA levels were exceedingly high for colon, spleen and lung. Further analysis of primary and metastatic mouse prostate cancer cell lines by Northern blotting indicated that RTVP was present at low levels in primary tumor-derived cell lines, but appeared to be down regulated in metastasis-derived cell lines. This indicated the possibility that loss of expression of this gene may be selected for during metastatic progression of prostate cancer.

To determine the potential functional significance of RTVP, adenoviral vectors containing the mouse RTVP cDNA were constructed and this adenoviral vector together with a control vector for apoptotic activity were tested in the p53 null human colon cancer cell line, H1299. The data clearly indicated that, at MOI of 100, more than 2-fold increase in annexin V positive cells was seen in RTVP infected cells relative to the same MO1 of a control virus that expressed only the beta-galactosidase gene. Additional studies were also consistent with RTVP stimulated apoptosis. These data indicated that a novel p53-regulated gene that is involved in apoptosis in prostate cancer and potentially other malignancies had been identified. That sequence, and the peptide it encodes, provide potential diagnostic/prognostic tools and/or therapeutic targets in clinical malignancies and growth disorders.

To further analyze the effects of RTVP-1 on the immune system RTVP negative mice were created using standard knock-out technology. Studies using homozygous RTVP negative mice in comparison to mice homozygous for the RTVP wild type indicated that loss of RTVP expression disrupted antigen presenting cell, specifically dendritic cell, activation and potential antigen presentation. Furthermore, studies of splenocytes from these RTVP negative mice demonstrated that CTL responses to allogenic cells were severely impaired. These results suggest that the inhibition of RTVP expression could be useful in the treatment and prevention of autoimmune disorders and graft rejections. Furthermore, these results suggest that the administration of RTVP, or active fragments thereof, could increase or stimulate an immune response providing useful treatments for infectious diseases.

Accordingly, one embodiment of the invention is directed to nucleic acids that comprises all or one or more portions of the sequence of RTVP, to peptides derived from these sequences, and to sequences complementary thereto. The nucleic acid sequence that encodes mouse RTVP-1 protein is shown in FIG. 1a (SEQ ID NO: 1). The amino acid sequence of the mouse RTVP-1 protein (SEQ ID NO. 2) is shown in FIG. 1b. Nucleic acids of the invention may be single-stranded or double-stranded and composed of DNA, RNA or PNA, or another appropriate nucleic acid, polypeptide or functionally similar backbone structure. Single stranded nucleic acids may be in the form of a sense strand or an antisense strand. Further, although the human RTVP gene sequence is know (GenBank DNA database accession number X91911), RTVP genes of the invention may be derived from other mammals such as, for example, mice, rats or any rodent, cattle, sheep, goats, pigs, horses, canines, felines, and most any other non-human mammal.

Nucleic acids according to the invention include isolated (e.g. purified) and recombinant nucleic acid sequences comprising SEQ ID NO: 1, or a portion of SEQ ID NO: 1 encoding all or one or more active fragments of the RTVP polypeptide. Nucleic acids containing conserved regions of sequences and nucleic acids encoding open reading frames and conserved domains within open reading frames are typically sufficient to represent or contain identifiable portions of RTVP such as functional and antigenic portions. Nucleic acids may comprise additional sequences such as RTVP-specific promoters, activator and repressor sites, and enhancers for modulation of expression of sense or antisense messages, recombination sequences for gene targeting, selectable markers for transfections, or replication origins for passage in a host such as bacteria, virus, eukaryotic cells or yeast. A further embodiment of the invention includes RTVP-specific promoters which modulation transcription (e.g. by differential methylation of promoter sequences) of RTVP in normal, pre-malignant and malignant cell. These promoters can be functionally coupled to anti-neoplastic genes to treat or prevent cell proliferative disorders such as, for example, tumors, prostate cancer, and metastatic disease or to treat or prevent infectious disease and autoimmune disorders. Nucleic acids may be packaged in a viral vector such as, for example, a retroviral, a vaccinia or an adenoviral vector. In one embodiment, the sequence may be part of a homologous recombination vector designed to recombine with another sequence. The invention further includes vectors comprising the nucleic acid sequences of the invention, polypeptides expressed by these vectors, and recombinant cells comprising these vectors.

Another embodiment of the invention is directed to all or an effective fragment of a RTVP polypeptide comprising all or part of the amino acid sequence encoded from the nucleic acid sequence of FIG. 1a (see FIG. 1b; SEQ ID NO. 2), such as, for example, exonic fragments, fragments encoded in open reading frames of RTVP genes, conserved domains of the polypeptide or the nucleic acid, and p53 binding sites. Specifically, the invention includes isolated polypeptides comprising all or active fragments of the polypeptide encoded in an RTVP gene, and monoclonal and other antibodies to these peptides.

Nucleic acids and polypeptides or proteins (RTVP and receptor proteins) according to the invention may be used as a diagnostic or therapeutic tool in the detection, treatment or prevention of diseases, such as neoplastic disorders (e.g. malignant tumors, metastatic disease), cell or tissue growth disorders, autoimmune disorders, or infectious diseases. For example, one embodiment of the invention is directed to diagnostic aids or kits for the detection of neoplasia in a patient. Detection kits may comprise RTVP nucleic acid sequences or RTVP polypeptides whose presence or absence in the sample would be indicative of the presence of a disease such as, for example, prostate cancer or a prostate metastasis, autoimmune disorders, or infectious diseases. Samples which can be analyzed include samples of biological fluids (e.g. blood, plasma, interstitial fluid, urine, cerebrospinal fluid) and samples of biological tissue (e.g. surgical biopsy).

Another embodiment of the invention is directed to antibodies specifically reactive against polypeptides and proteins and fragments thereof of the invention. Antibodies may be polyclonal or monoclonal of any isotype (e.g. IgA, IgD, IgE, IgG1, IgG2a, IgG2b, or IgM), or antibody fragments (e.g. Fab, Fv), humanized antibodies, or recombinant or synthetic antibodies (e.g. variable region fragments). Further, the invention comprises hybridomas that express antibodies specifically reactive against polypeptides of the invention.

Another embodiment of the invention is directed to a method for treating a neoplastic or growth disorder, autoimmune disorders, or infectious diseases comprising administering a pharmaceutically effective amount of a composition comprising an RTVP polypeptide, polypeptide fragment or ligand of a polypeptide. Such compositions may be anti-angiogenic, be used to modulate (e.g. increase or decrease) cytokine or other immune system regulatory proteins or their activity, induce apoptosis, and/or stimulate a cell or humoral response. For example, in cell growth disorders such as prostate cancer and other neoplasias, cytokine expression may be improperly tuned off (e.g. methylated) in malignant or pre-malignant cells. As such, these gene products may also be useful as a diagnostic for malignancy. Alternatively, compositions of the invention may be useful in, for example, auto-immune disease or graft rejections to turn on genes whose activity may be improperly reduced or turned off (e.g. by methylation). Further, polypeptides of the invention may stimulate apoptosis and are useful as therapeutics to treat and prevent neoplasia such as, for example, tumors, metastasis and any uncontrolled cell growth. A further embodiment of the invention comprises the RTVP promoters which are differentially modulated (e.g. by methylation) and regulate expression.

The polypeptide or polypeptide fragment, or a ligand to the polypeptide may be administered by injection, pulmonary absorption, topical application and delayed release. The composition may further comprise a pharmaceutically acceptable carrier such as water, alcohols, salts, oils, glycerols, fatty acids, starches, saccharides, polysaccharides or combinations thereof. More than one carrier may be used together to create a pharmaceutical with desirable properties. A further embodiment of the invention comprises vaccines for the treatment and/or prevention of neoplastic disease, autoimmune disorders or infectious diseases. Vaccines may comprise antibodies reactive against polypeptides and/or polypeptide fragments of the invention, of the polypeptides themselves. Vaccines comprise therapeutically effective doses of the therapeutic agent, which may be the polypeptide or polypeptide fragment, or an antibody or collection of antibodies which bind or are otherwise reactive thereto.

Another embodiment of the invention is directed to nucleic acids derived or based on the sequence of RTVP such as, for example, the sequence of SEQ ID NO: 1, useful in treatment or diagnosis and in diagnostic kits. Treatment may involve using the sequences, or effective parts thereof, in gene therapy, including gene ablation, gene expression and gene suppression, such as antisense suppression or siRNA expression. Diagnosis may involve genotypic analysis of samples to determine the existence and expression levels of the genes. Nucleic acids of the present invention may be used in various treatment and research modalities, including gene replacement, gene targeting, antisense inhibition, antisense blocking, siRNA inhibition, genetic ablation and gene silencing. Gene replacement involves replacing a copy of a defective gene with another copy by homologous recombination. Gene targeting involves the disruption of a cellular copy of a gene by homologous recombination. Gene targeting refers to a process of introducing a nucleic acid construct into a cell to specifically recombine with a target gene in the cell. The nucleic acid construct inactivates the gene after targeting. Inactivation may be by introduction of termination codons into a coding region or introduction of a repression site into a regulatory sequence. Antisense inhibition exploits the specificity of hybridization reactions between two complementary nucleic acid chains to suppress gene expression. If a cloned gene is engineered so that only the opposite DNA strand is transcribed, the resultant RNA may hybridize to the sense RNA and inhibit gene expression. Antisense blocking refers to the incorporation into a cell of expression sequences which direct the synthesis of antisense RNA to block expression of a target gene. Antisense RNA hybridizes to the mRNA of the target gene to inhibit expression. Genetic ablation (gene knockout) refers to one process of silencing a gene in a cell. Genetic ablation (gene knockout) may be performed after a cell is selected for use or by selecting a cell already comprising a genotype with the proper genetic ablation. Ablation of the gene encoding RTVP, for example by pre-transcriptional inhibition (such as homologous recombination with endogenous recessive oncogenes) or post transcriptional inhibition (such as the expression of antisense oncogenes to suppress translation) may be useful. Gene silencing is performed by transfecting cells with nucleic acids which cause genetic ablation or by antisense suppression. The silencing process may include processes such as gene targeting or antisense blocking.

Another embodiment of the invention is directed to methods to modulate a cytokine activity with effective amounts of RTVP protein or active portions thereof. Cytokines whose activity may be up regulated (e.g. through demethylation) include the type 1 or TH1 cytokines such as, for example, INF-alpha, beta and gamma, TNF-gamma, IL-2, IL-6, IL-12, and the death domain proteins such as Fas, and their ligands (i.e. receptors) such as Fas-ligand and Trail. Cytokines whose activity may also be modulated by RTVP include the type 2 or TH2 cytokines such as, for example, IL-1β, IL-4, IL-10. Increased or decreased expression or function of cytokines is an important aspect of RTVP therapy and has implications for both cancer and non-cancer therapy. One of the more important cytokines induced is IFN-gamma, wherein RTVP-1 is believed to be inducing a methylation change in the IFN-gamma promoter, which is normally completely methylated and therefore silent in normal cells. Differential methylation of these and RTVP promoter sequences can be used diagnostically to detect as well as therapeutically to treat neoplastic disease, autoimmune disorders or infectious diseases. Alternatively, in specific cancer cells including prostate cancer, global perturbations in methylation can silence tumor suppressor genes such as, for example, p16, and also lead to demethylation of genes that (i) produce a more malignant cell and is therefore selected during progression (e.g. such as caveolin-1), or (ii) make the cell susceptible to apoptosis when an appropriate stimulus is provided. In prostate cancer, the IFN-gamma gene promoter may be demethylated and therefore susceptible to induction. RTVP-1, which is expressed in normal prostatic epithelial cells, then becomes lethal to the cell by activating IFN-gamma which leads to direct cell killing or indirect cell killing via an immune response. Upon isolation of the RTVP receptor, ligands could be identified that could activate the RTVP-1 pathway, turn on IFN-gamma or another cytokine, which may be cancer and immune cell specific and thereby specifically target cancer cells.

Another embodiment of the invention is directed to receptor protein for RTVP proteins such as an RTVP-1 receptor, which is important for stimulating downstream activities of RTVP-1. Downstream activities includes apoptosis and stimulation of cytokine expression and apoptosis through stimulation of an immune response, which offers an extra-cellular therapeutic approach.

The following examples illustrate embodiments of the invention, but should not be viewed as limiting the scope of the invention.

### EXAMPLES

### Example 1

### Identification of mRTVP-1 as a p53 Target Gene

Over the last decade, numerous studies have established p53 mutations as being paramount to the development and progression of various malignancies. The p53 tumor suppressor protein has been associated with various anti-tumor activities that include growth suppression, apoptosis, and anti-angiogenic activities. The majority of human tumor mutations decrease the sequence-specific DNA binding and transcriptional activity of the p53 protein.²⁶ Today the large and expanding list of genes under the transcriptional control of p53 have been reported and it has been estimated that 200-300 genes are under the transcriptional regulation of p53.²⁷ To date, the number of reported p53-regulated genes is approximately 80.²⁸

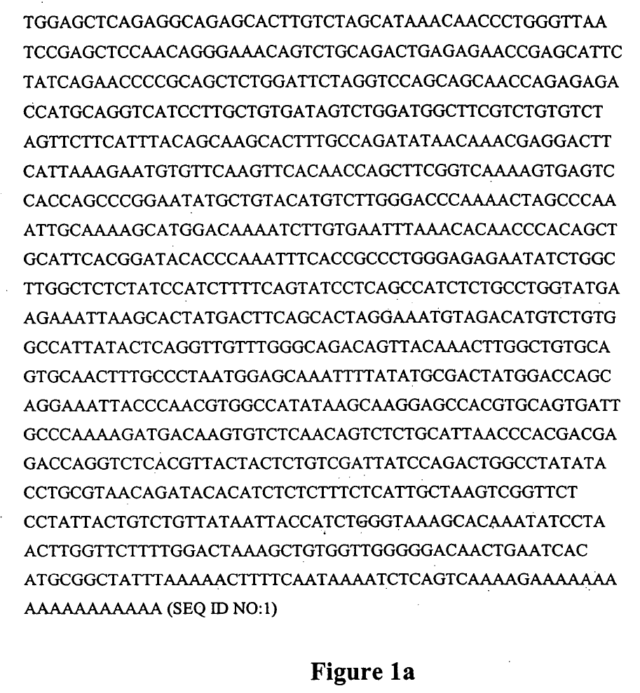

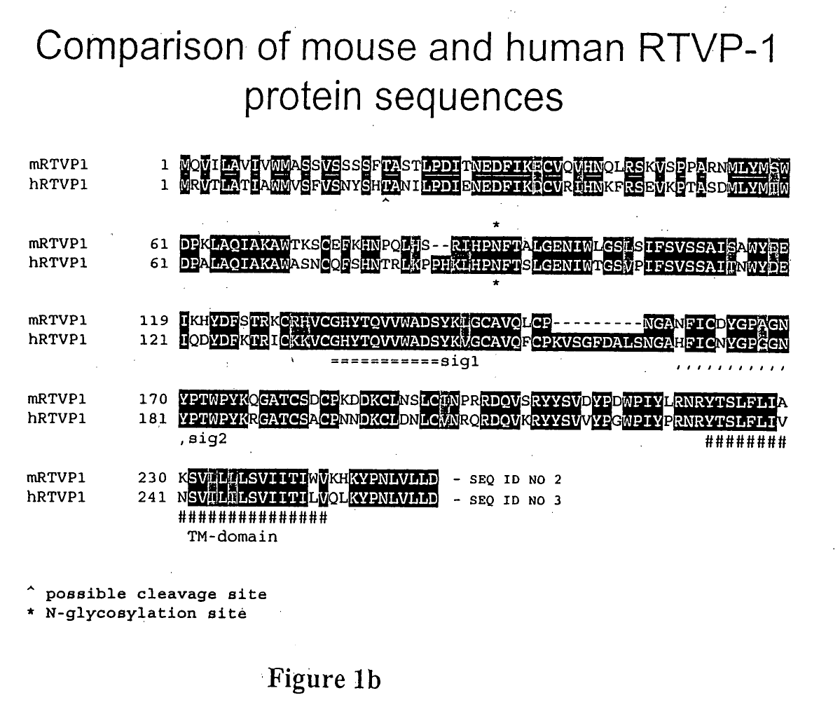

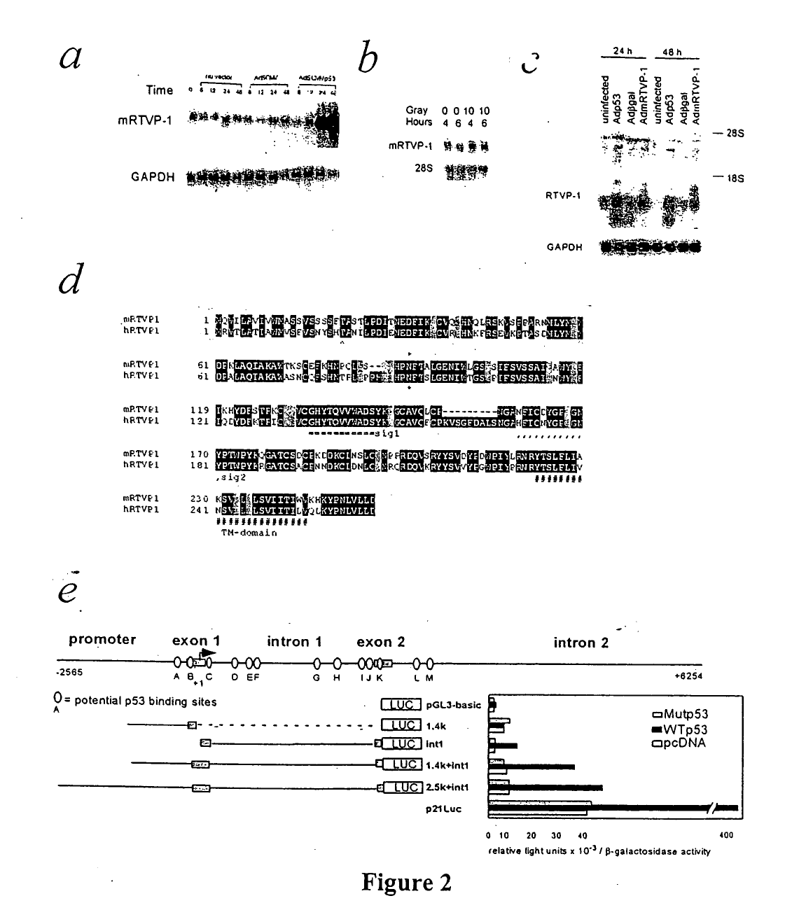

To identify prostate cancer-related genes under the transcriptional regulation of p53, a model system was established using adenoviral vector delivery of wild-type p53 compared to β-gal or an empty cassette together with differential display-PCR. Using this approach, numerous sequences were isolated that were known to be under p53 control including cyclin G, epoxide hydrolase, and MDM2. In addition, genes were isolated that had not been previously associated with p53 regulation. One of the sequences identified encoded the mouse homologue for RTVP-1.^{23,24} Briefly, mouse prostate cancer cell lines were grown in DMEM with 10% fetal bovine serum (FBS). Human prostate cancer cell lines Tsu-Pr1 and LNCaP were grown in RPMI 1640 with 10% FBS and PC3 in Kaighn's modified am's F12 with 10% FBS. For adenoviral infections the vector was diluted to the appropriate multiplicity of infection (MOI) in serum free medium with 0.1% bovine serum albumin and left in contact with the cells for 4 h followed by fresh media. Differential display-PCR was as previously described³⁴ using RNA isolated from AdCMVp53 or Ad gal infected 148-1PA cells. Differentially expressed PCR band was subcloned, sequenced and used to screen mouse and human cDNA and mouse genomic libraries. Mouse mRTVP-1 cDNA was inserted into the plasmid pcDNA3.1 (Invitrogen, Carlsbad, Calif.) for transfection studies or used to prepare an adenovirus (AdmRTVP-1) as previously described.^{35,36} Genomic DNA containing mRTVP-1 promoter fragments were subcloned into the luciferase reporter vector pGL3-luc (Promega, Madison, Wis.) and equimolar concentrations transfected into Tsu-PR1 cells with Lipofectamine along with mutant or wild type p53 expressing plasmids.³⁷ Luciferase activity was standardized to a cotransfected-galactosidase plasmid as previously described.³⁸ Freshly isolated rat aortic ring slices were infected with adenoviral vector in serum free endothelial medium (EGM-2, BioWhittaker, Walkersville, Md.) for 3 h then cultured in Matrigel for 48 h to allow for endothelial cell sprouting as described.³⁹ Protein lysates were prepared with TNES lysis buffer [50 mM Tris (pH 7.5); 2 mM EDTA, 100 mM NaCl, 1% NP40, 20 μg/ml aprotinin, 20 μg/ml leupeptin and 1 mM PMSF], separated on 10-12% polyacrylamide-SDS gels and electrophoretically transferred to nitrocellulose membrane for western blotting. Antibodies included Bad and phospho-Bad (New England Biolabs, Beverly, Mass.); BID (R&D Systems, Minneapolis, Minn.); Bax, Bcl-2, Bcl-X_{L}, caspase 6 and cytochrome c (BD-Pharmingen/Transduction Labs, San Diego, Calif.); and caspases 3, 7, 8, 9, and 10 (Oncogene Research Products, Boston, Mass.). β-actin monoclonal antibody (Sigma. St Louis, Mo.) was used as a loading control. Rabbit polyclonal antibody to peptides 59-71 of the mRTVP-1 protein was affinity purified. RTVP-1 cDNA was originally cloned from human glioma tissue and was subsequently reported to be expressed in differentiated macrophages.²⁵ Shown in FIG. 2 is the identification of RTVP-1 regulation by p53. Kinetic analysis of mRTVP-1 mRNA expression following infection of the p53 null mouse prostate cancer cell line 148-1 PA⁶ with no adenoviral vector, control adenoviral vector (Ad5CMV), or p53 expressing Ad5CMVp53 (FIG. 2a).⁴² Induction of mRTVP-1 by γ-irradiation in p53 wild type prostate cancer cell line RM9 (FIG. 2b).⁴³ Induction of RTVP-1 by Ad5CMVp53 in the human prostate cancer cell line Tsu-Pr1 relative to uninfected, control Ad βgal, or AdmRTVP-1 (FIG. 2c). Comparison of deduced protein sequence for mouse and human RTVP-1 (FIG. 2d). Identical amino acids are enclosed in dark boxes, conserved amino acid substitutions are lightly shaded, and gaps in the alignment by a −.A potential N-glycosylation site is indicated by a *. The putative signal sequence precedes a cleavage site indicated by the symbol \hat{ }. Two conserved regions known as extracellular protein signature motifs 1 and 2 (sig1 and sig2) and a transmembrane domain (TM) are indicated. Schematic of the genomic sequence with potential p53 binding sites and fragments used for luciferase constructs (FIG. 2e). The mRTVP-1 genomic sequence was from clone 163K10 from the mouse RPCI-21 PAC library.⁴⁴ DNA sequence analysis identified multiple p53 consensus binding sites (RRRCWWGYYY n RRRCWWGYYY, SEQ ID NO. 4; where R=purine, Y=pyrimidine, W=A or T and n=1-22 nucleotides) as well as recognition sites for IFNβ, NFκB, GM-CSF and AP-1 (not shown). The luciferase activity was determined 24 h after transfection of mRTVP-1-luciferase expression vectors into Tsu-Pr1 cells along with a β-gal plasmid for standardization and a plasmid expressing mutant p53 (shaded boxes), wild type p53 (solid boxes), or control plasmid (open boxes). A p21 promoter luciferase plasmid was used as a positive control for p53 activation.

Using the differential display-PCR fragment as a probe, it was determined that mRTVP-1 mRNA (˜1.1 kb transcript) was significantly induced at 24 and 48 hours after AdCMVp53 infection in the p53 null mouse prostate cancer cell line 148-1 PA and by γ-irradiation in mouse prostate cancer RM-9 cells that contain wild-type p53. Additional studies showed that RTVP-1 (˜0.8 and 1.3 kb transcripts) expression was also induced following AdCMVp53 infection in the human prostate cancer cell line Tsu-Pr1. Differential display-PCR fragment was used to screen a cDNA library generated from 148-1 LMD mouse prostate cancer cells and isolated multiple cDNAs containing the complete ORF of mRTVP-1 that encodes 255 amino acids with 68% identity to the human RTVP-1 protein. Notably, mRTVP-1 contains two short in-frame deletions of two amino acids (PH) at positions 86, 87 and a nine-amino acid deletion (KVSGFDALS SEQ ID NO.5) from amino acid 158 through 166 relative to human RTVP-1. Both mouse and human proteins contain a putative N-linked glycosylation site and a hydrophobic region near the carboxy terminus. Interestingly, both proteins also contain putative N-terminal signal peptides and extracellular protein signature motifs, which suggests that both proteins are potentially located on the surface of the cell membrane or even secreted. The mRTVP-1 cDNA was also used to isolate genomic mRTVP-1 and sequenced 2.5 kb of promoter sequences as welt as exon 1, intron 1 and exon 2 of the mRTVP-1 gene. Multiple putative p53 binding sites were documented in the mRTVP-1 promoter and intron 1, with at least 13 in intron 1. In co-transfection studies using mRTVP-1-luciferase expression vectors, specific induction of luciferase activity by exon 1-intron 1 sequences following co-transfection mRTVP-1-Int1-luc with wild-type p53 in Tsu-Pr1 cells was demonstrated. When 1.4 or 2.4 kb of promoter sequences was included upstream of exon 1-intron 1, a significant increase in p53 inducibility, but also basal promoter activity was observed. These results suggest that one or more putative p53 binding sites within intron 1 are mediating p53 regulation of mRTVP-1.

### Example 2

### RTVP-1 mRNA Levels are Down-Regulated in Prostate Cancer Metastases

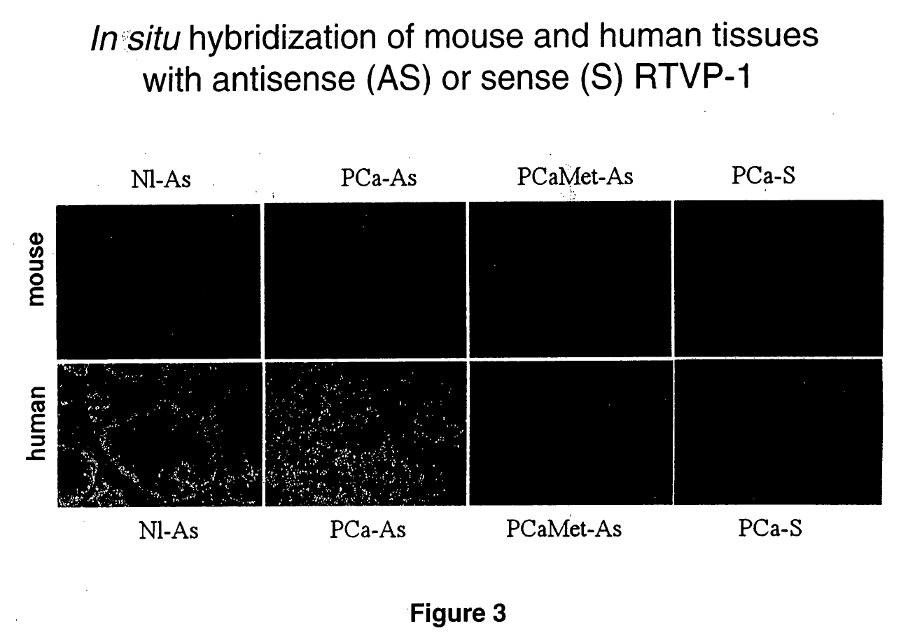

Having demonstrated that mRTVP-1 is induced by p53; it was next determined the mRNA expression profile of RTVP-1 in mouse and human prostate cancer tissue specimens by in situ hybridization (FIG. 3). Briefly, normal mouse prostate or prostate cancer developed in the mouse prostate reconstitution model with strain 129/SV wild type, heterozygous or homozygous for p53 knockout⁶ were used for in situ hybridization. Human primary prostate cancers and lymph node metastatic deposits as well as histologically normal prostate were obtained at radical prostatectomy. The specimens were frozen in liquid nitrogen immediately after surgical removal and 6-μm sections cut with a cryostat. Sections were air-dried and fixed in a solution containing 3 parts 4% paraformaldehyde, 4 parts ethanol and 3 parts glacial acetic acid for 20 min. Anti-sense cRNAs of mRTVP-1 (400 bases) and hRTVP-1 (256 bases) or corresponding sense RNA probes were made by run-off transcription of the vector pCR2.0 (Invitrogen) with SP6 or T7 polymerase, respectively using the DIG-RNA labeling kit (Roche Diagnostics Corp. Indianapolis, Ind.). The sections were prehybridized in 50% formamide, 5×SSC, 5× Denhardt's solution, 250 μg/ml yeast t-RNA, 4 mM EDTA and I mg/ml salmon sperm DNA at 37° C. for 60 mm and hybridized in the prehybridization buffer (without the salmon sperm DNA) containing 2.5 to 10 ng/μl DIG-labeled cRNA probes at 48° C. overnight. Sections were then rinsed in 4×SSC (2×10 min), 2×SSC (10 min) and 1×SSC (10 min) and 0.1×SSC (30 min at 48 C). The DIG-labeled RNA was detected with mouse anti-DIG IgG followed with a DIG conjugated antibody to the mouse IgG Fab fragment and finally anti-DIG IgG conjugated with fluorescein (Roche). Sections from normal prostate and cancer as well as metastatic cancer deposits were always processed in parallel under the same conditions and using the same batches of probes and reagents. Sections were evaluated under a fluorescence microscope and the RTVP-1 mRNA levels were scored according to the relative fluorescence intensities (see Table 2) as −: No signal detectable, +: weak; ++ moderate, and +++: strong. Statistical analysis using Mann-Whitney U test was performed to determine the significance of the differences in the fluorescence scores in different tissues.

Shown in FIG. 3 is RTVP-1 mRNA expression in mouse (m) and human (h) prostatic tissues as demonstrated by in situ hybridization with fluorescent riboprobes. RTVP-1 mRNA is expressed in the basal and glandular epithelial cells of both normal mouse and human prostates (mNP-AS or hNP-AS respectively). Cancer cells expressed a moderate level of RTVP-1 mRNA in both mouse (mPCa-AS) and human (hPCa-AS) primary prostate tumors. In contrast, much lower levels of RTVP-1 mRNA were shown in the metastatic deposits of both mouse (niPCaMet AS) and human (hPCa-Met AS) lymph nodes. Sections from both mouse and human prostate cancers that were incubated with the sense riboprobes gave rise to minimal signal (mPCa-S and hPCa-S). Original magnification: 200×.

Semi-quantitative in situ hybridization analysis of a panel of mouse (not shown) and human prostate cancer tissue specimens revealed abundant RTVP-1 mRNA levels in normal human prostatic basal and secretory epithelial cells and in primary tumor cells (Table 2). However, RTVP-1 mRNA levels were significantly reduced in metastases relative to normal prostatic epithelium or primary prostate cancer cells. Immunohistochemical analysis for p53 protein using the criteria of clustered staining¹⁰ revealed a general trend toward association of reduced RTVP-1 mRNA and aberrant p53 protein accumulation, yet the data were more consistent with independent inactivation of RTVP-1 (Table 2). Extensive sequencing analysis of RTVP-1 cDNAs isolated from prostate cancer cell lines and tissues did not reveal RTVP-1 gene mutations suggesting other mechanisms of gene inactivation (data not shown).

These data show that endogenous RTVP-1 mRNA expression is specifically reduced in metastatic mouse and human prostate cancer. Interestingly, RTVP-1 mRNA levels were abundant in normal prostate epithelial cells and in primary prostate cancer cells suggesting that the RTVP-1 gene is active in the absence of high levels of p53 protein. Therefore, it appears that p53-independent pathways can regulate RTVP-1 expression under both normal and abnormal conditions. Immunostaining analysis of p53 protein showed a trend towards an association between aberrant accumulation and reduced RTVP-1 mRNA levels yet the data were more consistent with independent inactivation of RTVP-1 gene expression. Initial sequencing analysis of cDNAs isolated from malignant prostate cancer cells failed to reveal any structural mutations in the RTVP-1 coding region.

### Example 3

### Pro-Apoptotic Activities of mRTVP-1 are Mediated Through the Mitochondrial Pathway

To analyze the biological activities of mRTVP-1, a series of human prostate cancer cell lines (Tsu˜Pr1, LNCaP, and PC3) and the human lung cancer cell line, H1299, were infected with AdmRTVP-1 or Adβgal. Western blotting analysis demonstrated high levels of intracellular mRTVP-1 protein by 48 hours after infection.

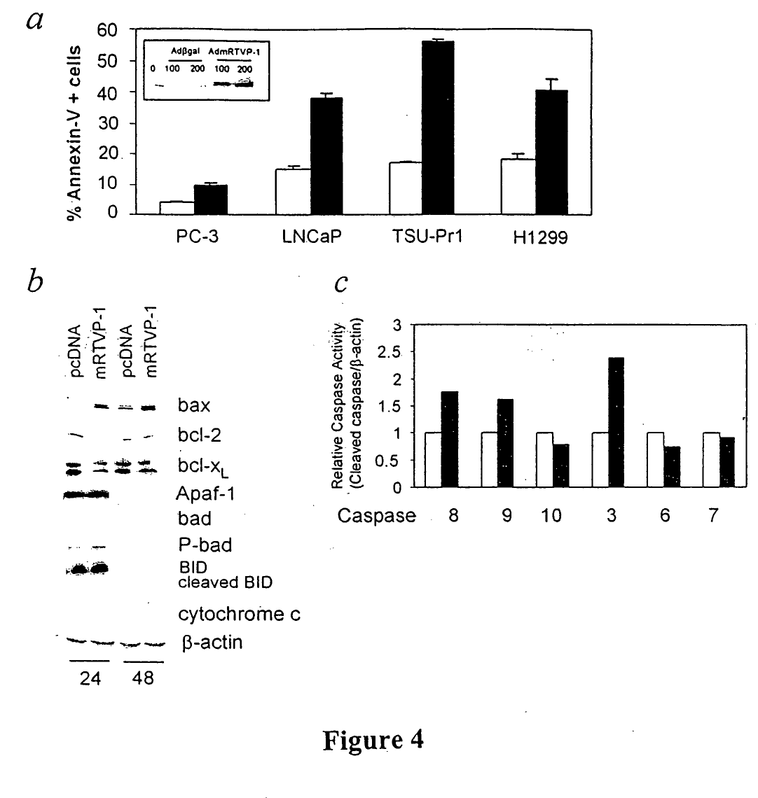

As shown in FIG. 4, mRTVP-1 induces apoptosis through mitochondrial death pathway. Apoptosis was determined by annexin V binding and flow cytometry on cells infected with Adβgal (open boxes) or AdmRTVP-1 (closed boxes) at an MOI of 100 as described (FIG. 4a).³⁸ Protein levels of mRTVP-1 in Tsu-Pri1 cells were determined by western blotting. Western blotting for bcl family member proteins and cytosolic cytochrome c in LNCaP lysates following transfection with control plasmid pcDNA or pmRTVP-1 (FIG. 4b). Relative caspase activity was determined by densitometric analysis of western blots of the cleavage product for each specific caspase relative to β-actin as a loading control in lysates from LNCaP cells following infection with Adβgal (open boxes) or AdmRTVP-1 (closed boxes) (FIG. 4c).

Thus, AdinRTVP-1 induced apoptosis as evidenced by significant increases in annexin V positivity, DNA fragmentation (not shown) and DAPI staining (not shown). To further probe the mechanisms of apoptosis induction by mRTVP-1, the expression of bcl-2 family proteins in LNCaP cells was evaluated following mRTVP-1 transfection. These results demonstrated that mRTVP-1 expression led to increased levels of pro-apoptotic bax, bad and activated BID, but down regulation of anti-apoptotic bcl-2 and bcl-X_{L}. Increased levels of cytosolic cytochrome c were also demonstrated following mRTVP-1 transfection, indicating activation of the mitochondrial apoptosis pathway. The activation of specific caspases was also analyzed using western blotting with antibodies specific for the activated forms of the initiation caspases 8, 9, and 10 and effector caspases 3, 6 and 7. The results indicated that AdmRTVP-1 infection was capable of inducing activation of caspases 8, 9 and 3 relative to control Adβgal infection in LNCaP cells. Following mRTVP-1 transfection, apoptosis was also induced with frequencies and characteristics similar to that observed following infection with AdmRTVP-1 (not shown).

### Example 4

### Adenoviral Vector-Mediated mRTVP-1 Expression Suppresses Prostate Cancer Growth and Metastasis

To determine possible therapeutic activities induced by overexpression of mRTVP-1 in vivo, an orthotopic mouse model of metastatic prostate cancer was used. Briefly, orthotopic tumors were initiated by injecting 5000 178-2BMA cells into the dorsolateral prostate of 129/SV mice. Seven days later when the tumors achieved an average wet weight of 25 mg they were injected with adenoviral vector in a volume of less than 25 μl. Six to ten animals were injected with each dose. At the indicated times, tumor tissue was weighed then frozen in liquid nitrogen or fixed in 10% formalin and processed for paraffin embedding. Apoptosis was determined by TUNEL labeling as previously described.⁴⁰ Mean vessel density and tumor cell infiltrate quantitation was essentially as previously described⁴¹ using monoclonal rat-anti mouse antibodies for CD8, CD4, F4/80, TNF-(BD-Pharmingen), as well as polyclonal antibodies to NOS2 (BD-Pharmingen) and Factor VIII-related antigen (Dako, Carpinteria, Calif.). Spontaneous metastases to the lung were microscopically counted after overnight fixation in Bouin's solution.⁴¹ NK activity was determined by lysis of YAK cells by splenocyte derived cells as previously described.⁴¹ Serum IL-12 was determined by immunoassay (Biosource, Camarillo, Calif.). All mice were maintained in facilities accredited by the American Association for Accreditation of Laboratory Animal Care and all experiments conducted in accordance with the principles and procedures outlined in the National Institutes of Health's Guide for the Care and Use of Laboratory Animals.

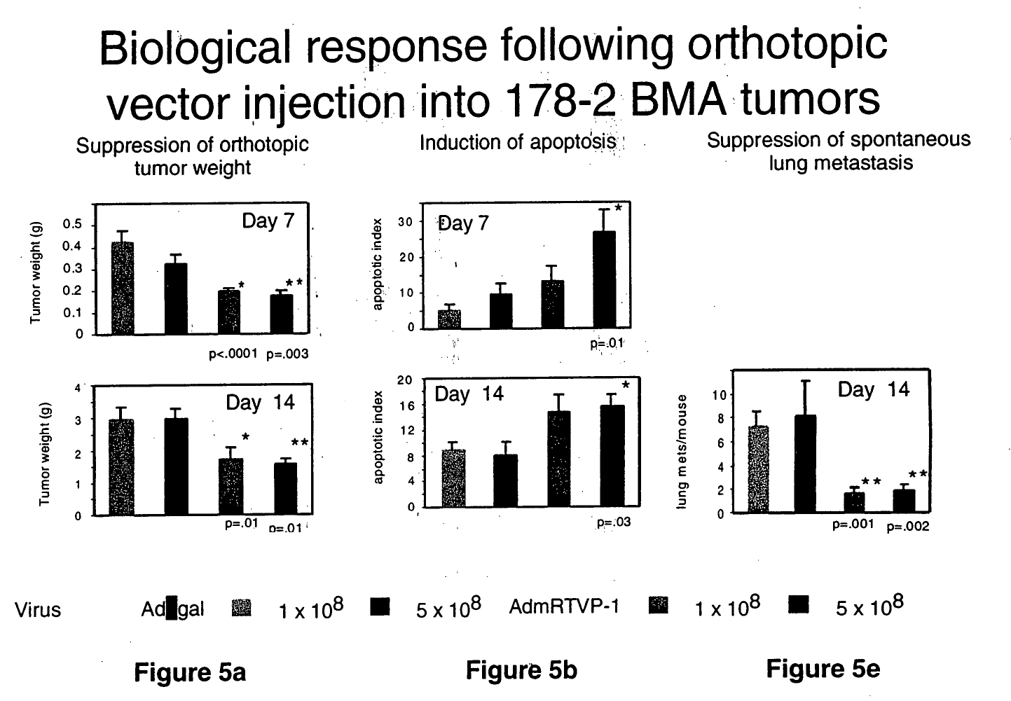

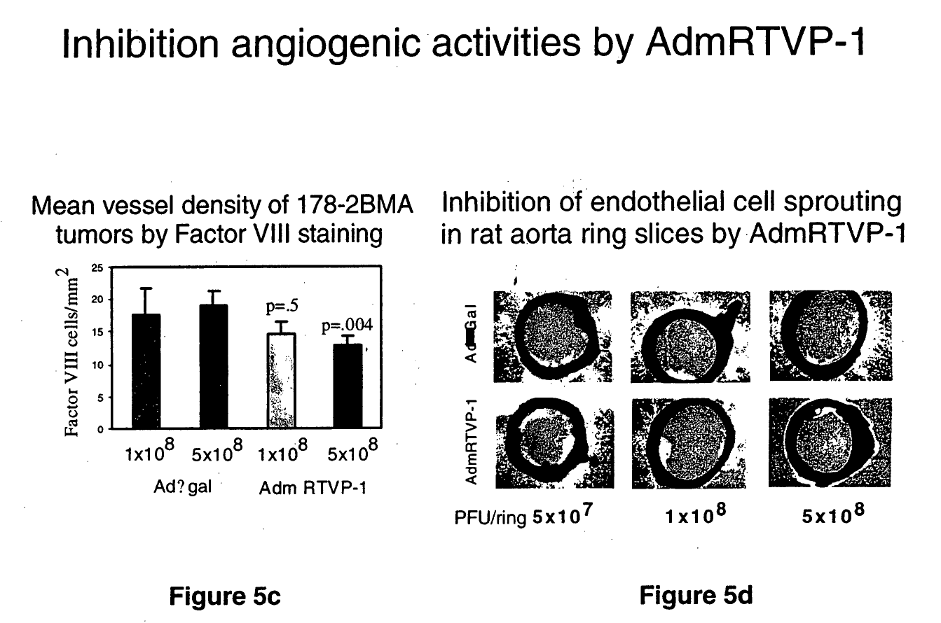

As indicated, seven days following orthotopic inoculation of 178-2BMA cells, tumors were injected with either 1×10⁸ or 5×10⁸ PFU of AdmRTVP-1 or Adβgal. At 7 and 14 days post vector treatment, tumors were recovered from animals and both primary tumors and their metastases were analyzed extensively. As shown in FIG. 5, mRTVP-1 suppresses tumor growth and metastasis. Wet weight of orthotopic 178-2BMA tumors treated with Adβgal at 1×10⁸ PFU (open box) or 5×10⁸ PFU (hatched box) or with AdmRTVP-1 at 1×10⁸ PFU (shaded box) or 5×10⁸ PFU (closed box) on day 7 (left panel) or day 14 (right panel) after tumor cell inoculation. * P≦0.05; ** P≦0.01 (FIG. 5a). Apoptotic index as determined by TUNEL labeling (symbols as in a) (FIG. 5b). The tumor microvessel density (symbols as in a) as measured by factor VIII staining was decreased by high dose AdmnRTVP-1 (FIG. 5c). Rat aortic ring slices were incubated with the indicated dose of adenoviral vector then placed in Matrigel and 48 h later photographed (FIG. 5d). Spontaneous lung metastases from adenoviral vector treated orthotopic tumors in animals was determined on day 21 (symbols as in a) (FIG. 5e).

AdmRTVP-1 significantly suppressed the growth of primary tumors compared to control Adβgal vector injections at both vector doses when evaluated at both time points (P≦0.01). Additional analysis indicated that increased apoptotic levels were likely responsible for the growth suppressive effects of AdmRTVP-1 as significant increases in TUNEL labeling were documented in tumors treated with 5×10⁸ PFU of AdmRTVP-1 at both day 7 (P=0.028) and day 14 (P=0.015). Further, a significant reduction (P=0.004) in the density of Factor VIII-positive tumor associated endothelium was also associated with the higher dose of AdmRTVP-1 when day 14 tumors were analyzed. In an independent in vitro angiogenesis assay, the rat aortic ring sprouting assay, treatment with AdmRTVP-1 inhibited endothelial cell sprouting compared to Adβgal. To evaluate potential antimetastatic effects of mRTVP-1 in the I 78-2 BMA orthotopic model, the extent of lung metastases in AdmRTVP-1-treated and control Adβgal-treated animals was analyzed on day 14 after tumor initiation. Both doses of AdmRTVP-1 significantly suppressed spontaneous lung metastases compared to Adβgal infection (P≦0.002), indicating that mRTVP-1 associated activities lead to suppression of growth and spontaneous metastatic activities of orthotopically grown mouse prostate cancer. These results suggest that mRTVP-1 mediated pro-apoptotic and anti-angiogenic activities likely played a role in suppression of tumor growth and metastasis.

### Example 5

### Expression of mRTVP-1 Induces a Local and Systemic Anti-Tumor Immune Response

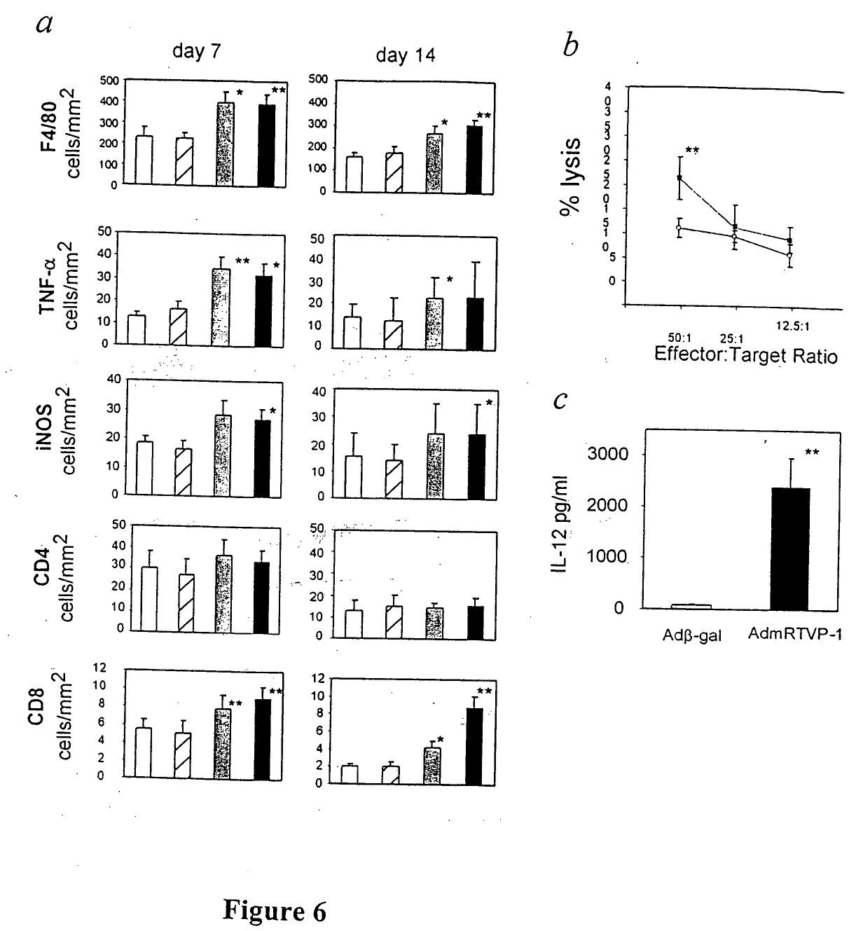

To further explore the underlying mechanisms responsible for mRTVP-1 mediated antimetastatic activities, local and systemic immune responses was analyzed in animals with AdmRTVP-1 treated tumors compared to control Adβgal infected tumors. Local and systemic immune response in AdmRTVP-1 treated tumors are shown in FIG. 6. Immunohistochemical detection of F4/80, TNF-α, NOS2 (iNOS), CD4 and CD8 positive cells were quantitated by image analysis and expressed as positive cells per mm² (symbols as in FIG. 5) (FIG. 6a). NK activity two days after vector inoculation. (▪=AdmRTVP-1 treated animals, ◯=Adβgal treated animals) (FIG. 6b). Serum IL-12 levels from animals sacrificed on day two or three after vector inoculation (FIG. 6c).

The results indicated significant increases in the density of tumor-associated F4/80-positive macrophages (P=0.03 for 1×10⁸ PFU and P=0.01 for 5×10⁸ PFU) and CD8⁺ T cells (P=0.004 at both doses) in AdmRTVP-1 infected tumors relative to control Adβgal infected tumors at both day 7 following vector treatment. Significant infiltrates of tumor associated macrophages and CD8+ T cells persisted for both doses at 14 days post treatment (P=0.017 and P=0.02 for macrophages and P=0.018 and P=0.0005 for CD8+ T cells for 1×10⁸ PFU and 5×10⁸ PFU respectively) indicating sustained immunostimulatory activities. Analysis of activation markers associated with tumor associated F4/80-positive macrophage indicated that AdmRTVP-1 specifically increased NOS-2 and TNF-α activities in this cell type. To determine whether mRTVP-1 overexpression in the primary tumor elicited a systemic immune response splenocytes were isolated from mice bearing AdmRTVP-1 and control Adβgal infected tumors and used for analysis of NK activities. Significantly increased NK activities were demonstrated two days post vector injection in the mice with AdmRTVP-1 treated tumors compared to mice with Adβgal treated tumors (P=0.01, unpaired t test at E:T of 100:1 and 0.004 at 50:1). In addition, a significant increase in serum levels of IL-12 (P=0.001) was seen in animals with AdmRTVP-1 treated tumors relative to Adβgal two to three days following vector injection.

Overall, these results specify that mRTVP-1 as a p53-induced gene and is involved in apoptosis-mediated tumor suppressor activities. Although there are numerous studies that have documented the tumor suppressing activities of p53 in various systems and clear evidence has been found for a role for p53 as a tumor suppressor gene in human cancer, the reported antimetastatic activities of p53 have thus far remained somewhat ambiguous. Certainly, the induction of apoptosis and suppression of angiogenesis could inhibit metastatic spread, yet in some malignancies such as prostate cancer the clear association of loss of p53 function with metastasis suggests the existence of additional p53-mediated anti-metastatic activities. These results show an additional role for p53 as an antimetastatic gene as an inducer of mRTVP-1, which in turn can generate an antitumor immune response that is manifest locally as well as systemically. One previously published report indicated that p53 could induce the CX3C chemokine, fractalkine, raising the possibility that p53 effector genes are also involved in immune cell stimulation.²⁹ It is of interest that RTVP-1 and fractalkine share some structural characteristics as predicted by the amino acid sequence. Both proteins have a consensus signal peptide and a putative transmembrane domain region. As fractalkine has been shown to be present in a soluble form^{30,31} it appears that this chemokine and potentially RTVP-1 exist as both membrane bound and soluble forms, and are therefore capable of paracrine activities. In the case of fractalkine, paracrine functions appear to be limited to chemotactic and pro-adhesion activities.^{30, 31} However, the paracrine functions of mRTVP-1 appear to generate widespread immunostimulatory activities in vivo that include the induction of F4/80-positive macrophage and CD8+ T cell infiltrates within the primary tumor; the induction of splenocyte derived NK activities; and the generation of increased serum IL-12 levels.

The uptake of increased numbers of apoptotic bodies by antigen presenting cells could lead to the activation of specific immune cells including macrophages that were shown to be increased within primary tumors following AdmRTVP-1 treatment. Activated macrophages or potentially activated dendritic cells could secrete IL-12 (shown to be increased in serum) and lead to increased systemic NK activities. Alternatively, membrane bound or soluble mRTVP-1 could directly activate immune cell activities. Since RTVP-1 expression has been associated with differentiated macrophages,²⁵ a role for RTVP-1 as a cytokine-like molecule is likely.

These data clearly demonstrate that p53 can induce expression of RTVP-1 in prostate cancer cells in vitro and that overexpression of niRTVP-1 has widespread immunostimulatory activities in vivo. Further, stress-related p53 induction may induce RTVP-1 levels sufficiently to induce immune cell activities that counteract the growth and progression of human prostate cancer.

These studies are relevant for prostate cancer as the development of effective treatments for prostate cancer are frustrated by the natural history of the disease. The biological and clinical potential of most individual cancers is uncertain and in many cases the disease will not progress to clinical significance. However, experimental and clinical studies indicate that prostate cancer can and may metastasize early in the course of the disease from relatively small foci.^{32,33} Although localized prostate cancer is potentially curable with radical prostatectomy or irradiation therapy, there are no curative therapies for metastatic prostate cancer. As shown by these results, expression of mRTVP-1 in prostate cancer through adenoviral vector transfer can induce local cytotoxicity through direct and indirect apoptosis while simultaneously initiating a local and systemic anti-tumor immune response. Thus, RTVP-based therapy may be useful as a new weapon against both prostate cancer and metastatic disease.

### Example 6

### Mechanism of Action of RTVP-1

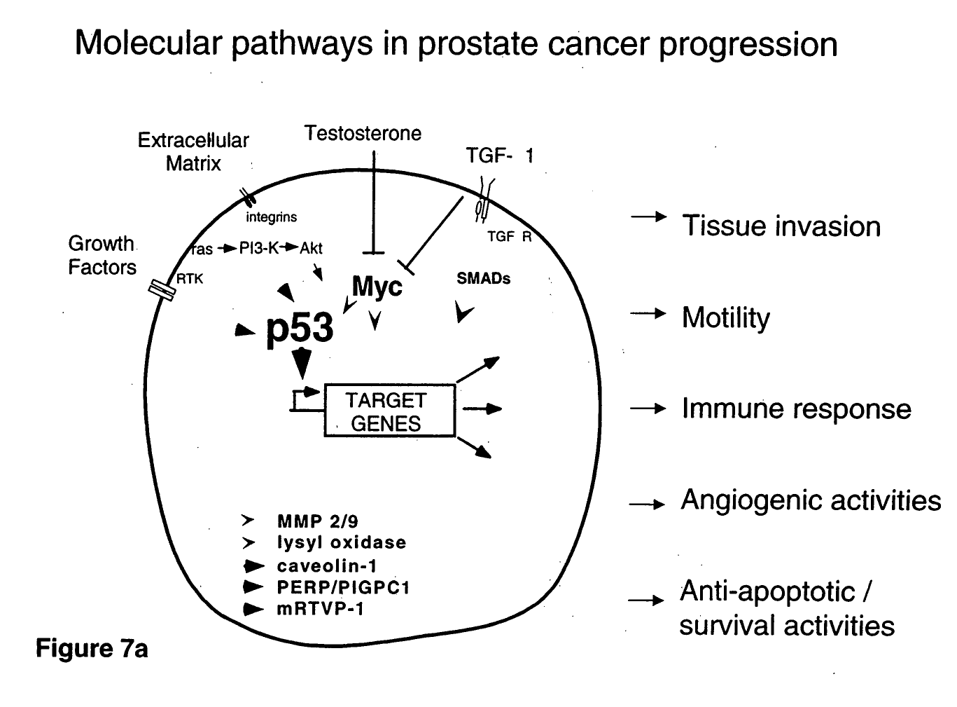

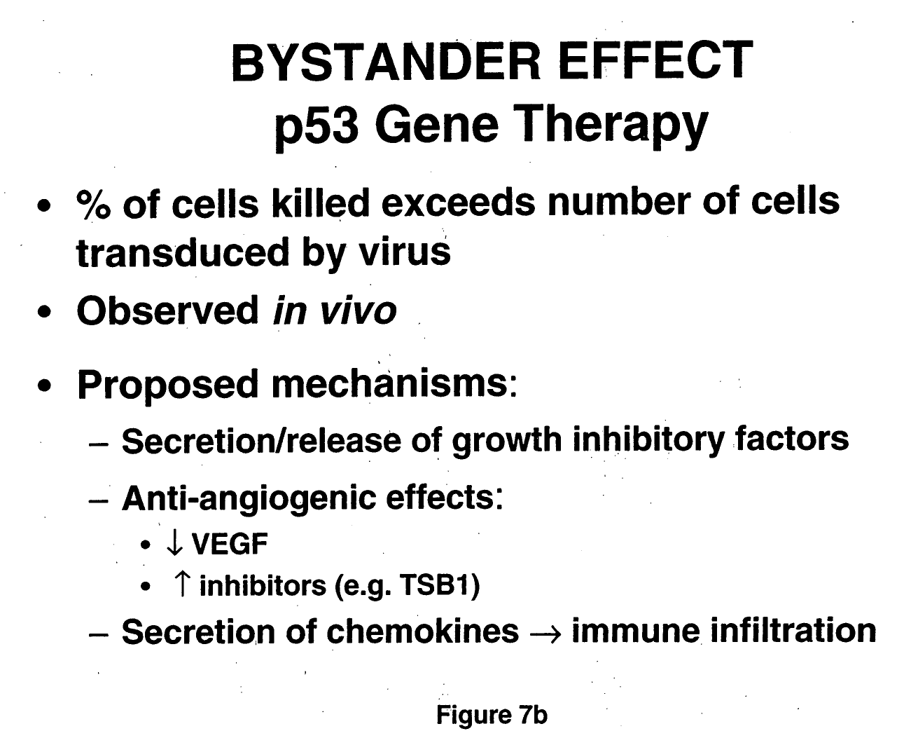

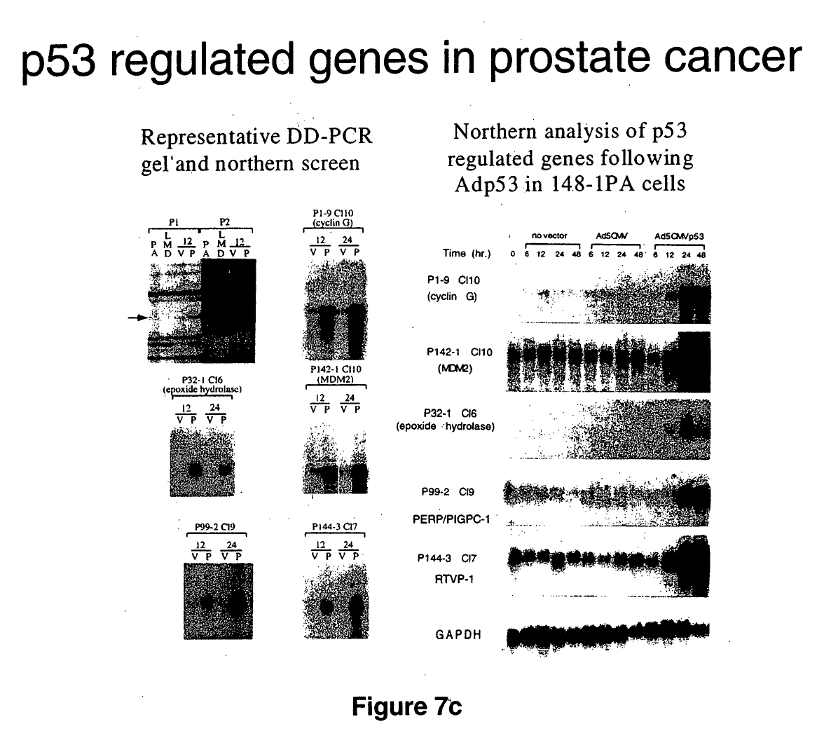

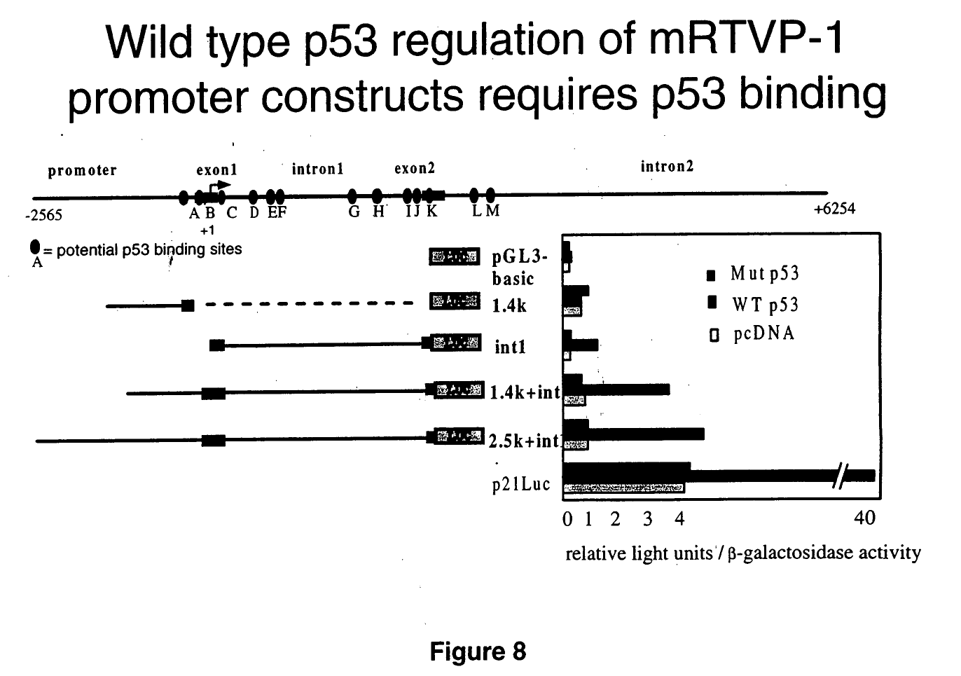

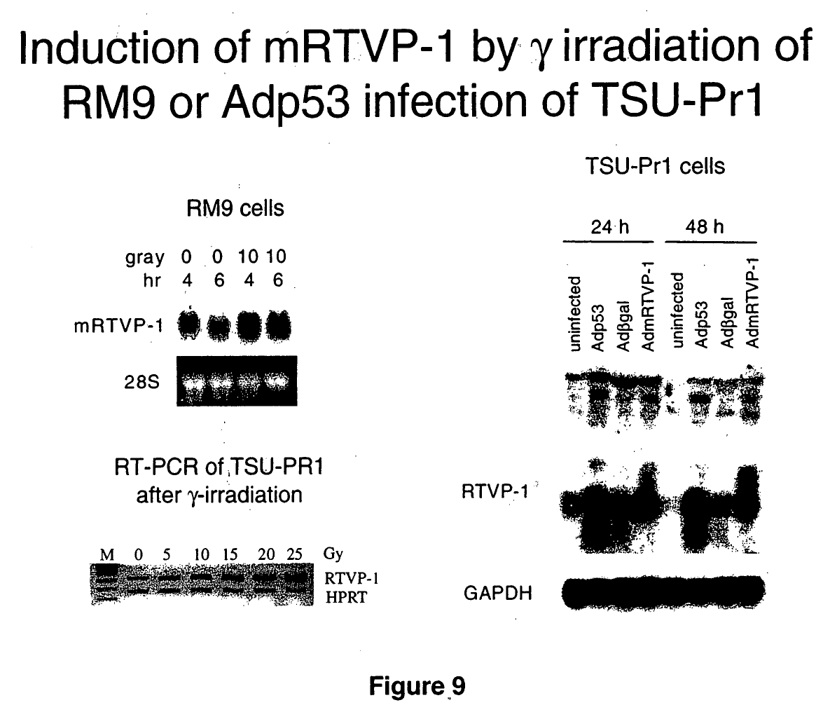

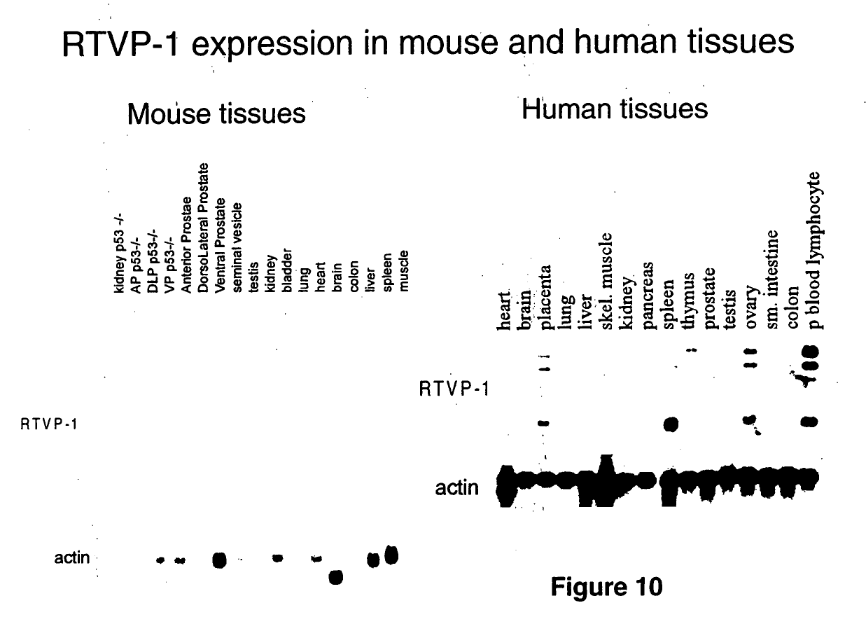

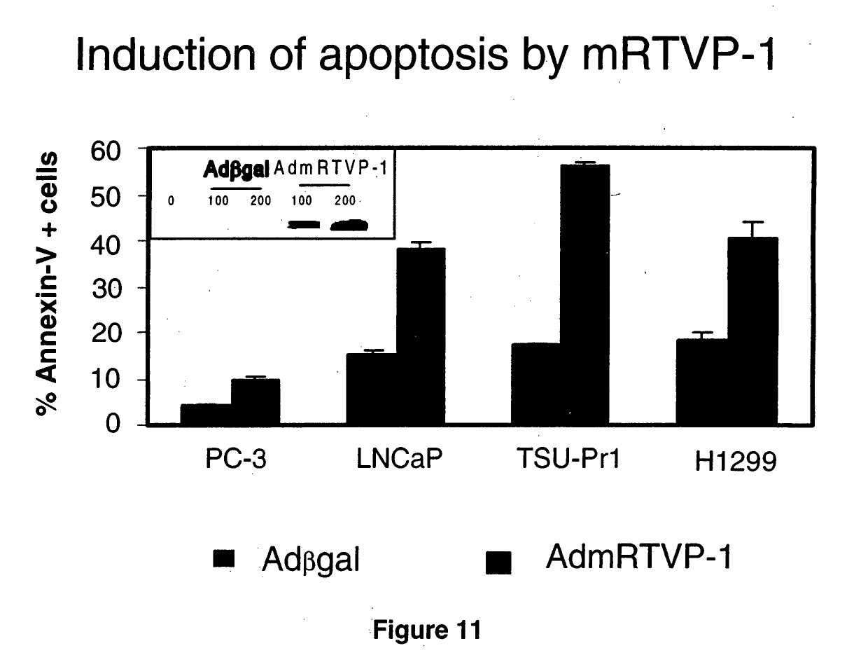

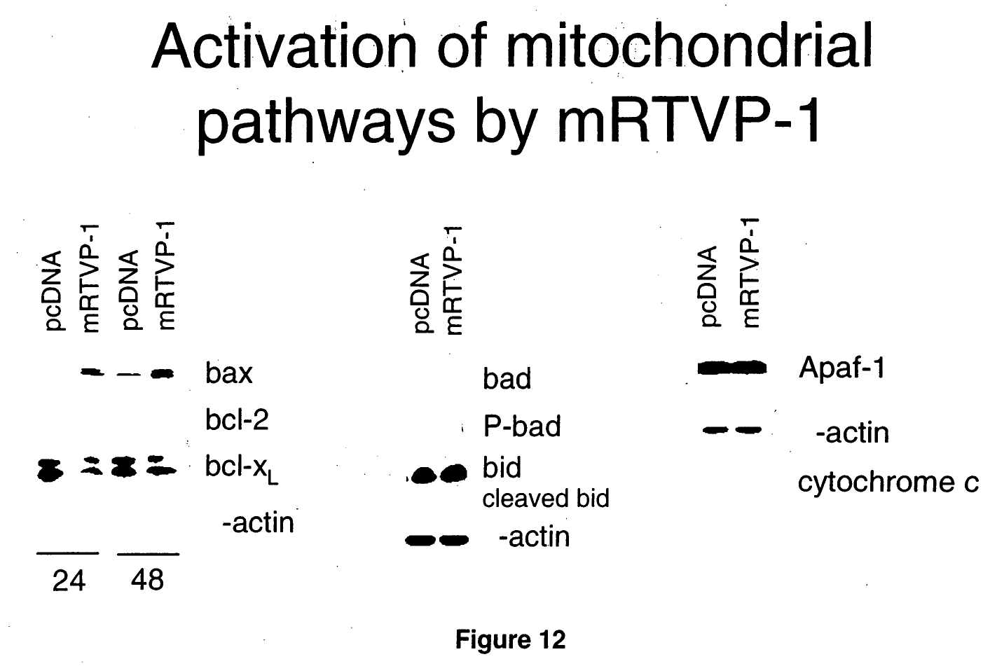

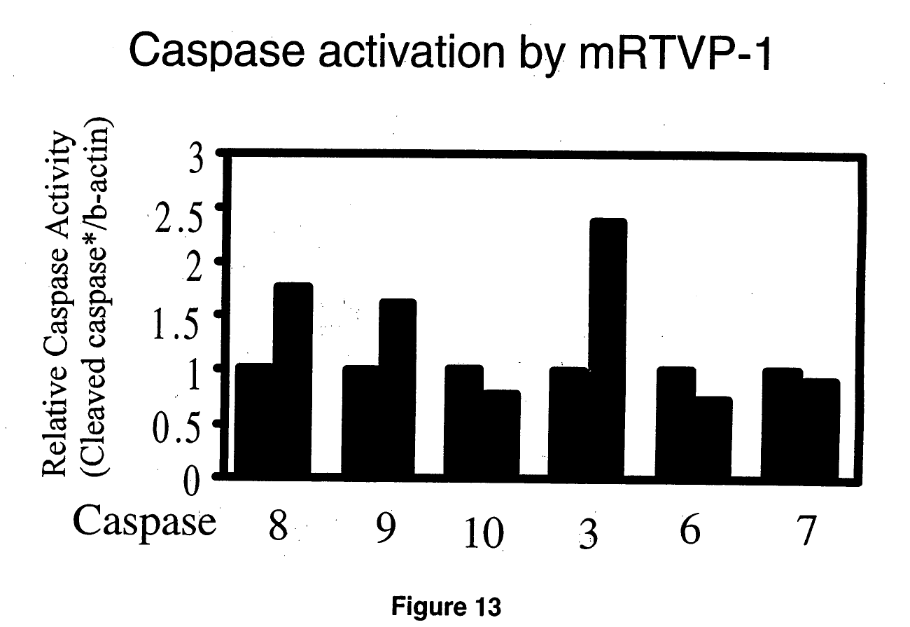

To further explore the underlying mechanisms responsible for mRTVP-1 mediated anti-neoplastic activity, additional experiments were conducted. All experiments were performed as indicated or according to procedures which are well known to those of ordinary skill in the art. As shown in FIG. 7, p53 has an important role in the progression of prostate cancer (FIG. 7a), both for a bystander effect (FIG. 7b), and for regulation of genes in prostate cancer (FIG. 7c). Promoter constructs of both mutant and wild-type p53 demonstrate that regulation of the MRTVP-1 promoter require p53 binding (FIG. 8). Further, induction of mRTVP-1 was shown in both RM9 and TSU-Pr-1 cells by Adp53 by gamma irradiation (FIG. 9). RTVP-1 expression was evaluated for both mouse and human tissues (FIG. 10). Induction of apoptosis was shown in PC-3 cells, LNCaP cells, TSU-Pr1 cells and H2299 cells (FIG. 11), as well as activation of mitochondrial pathways by mRTVP-1 (FIG. 12), and caspase activation (FIG. 13).

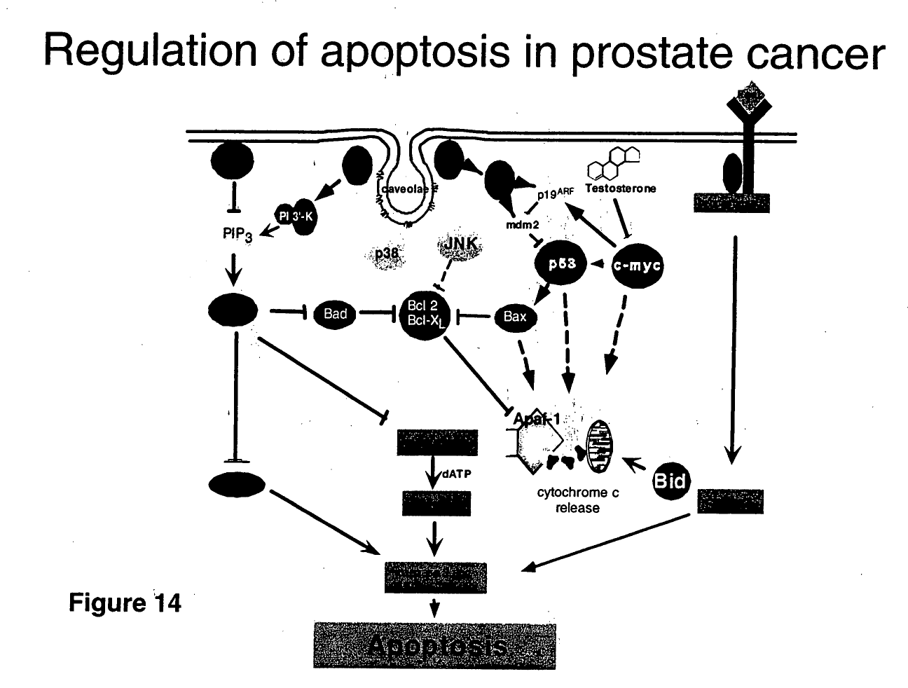

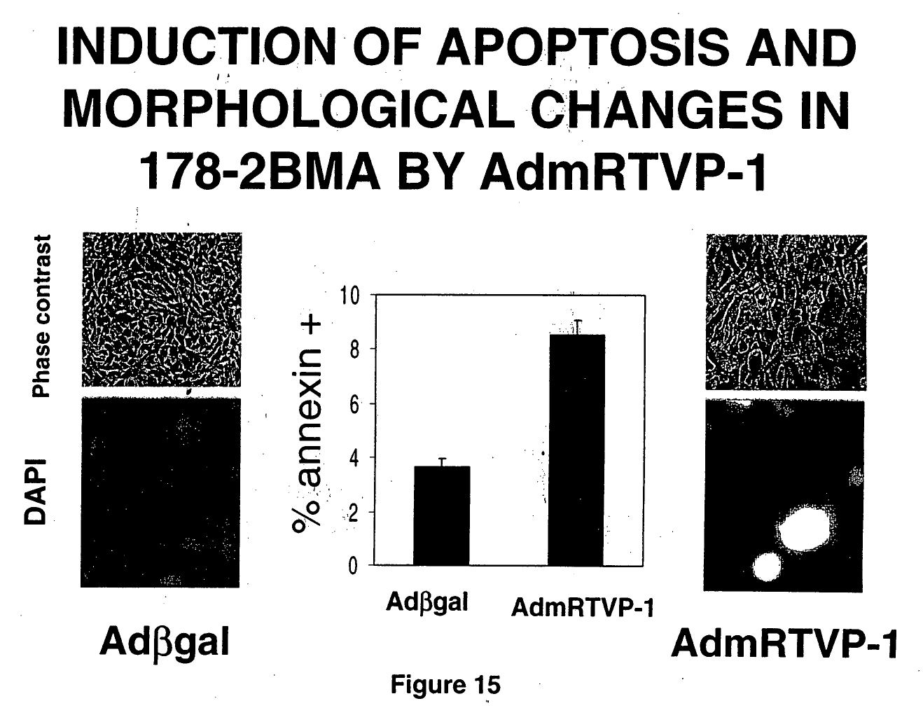

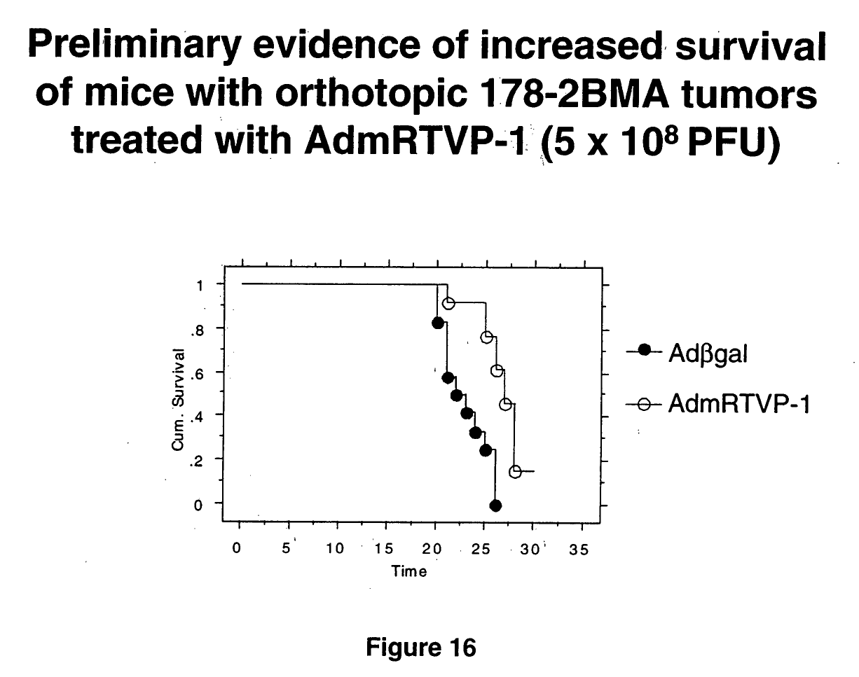

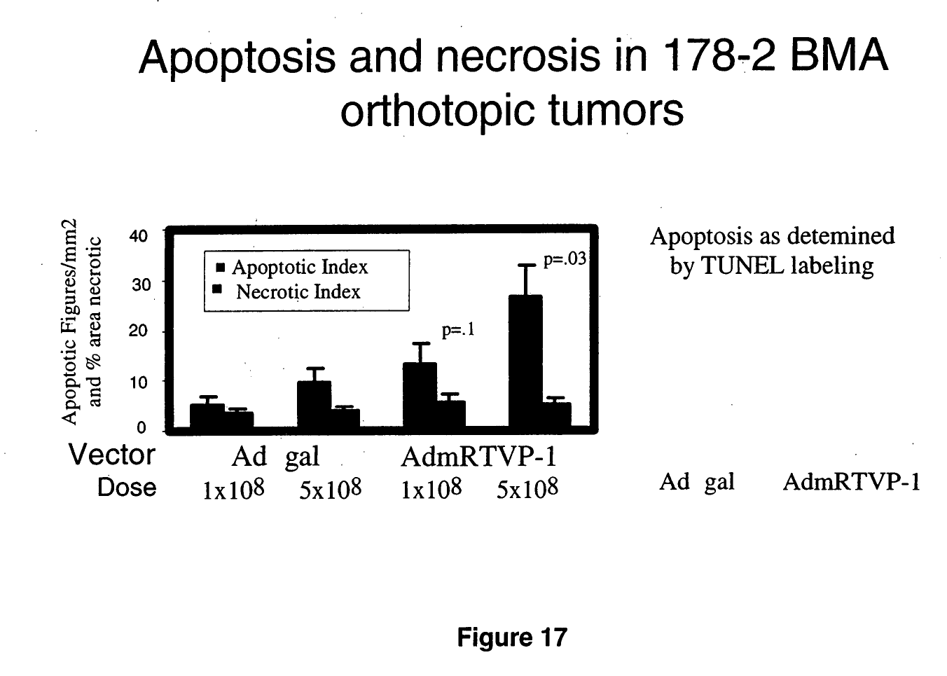

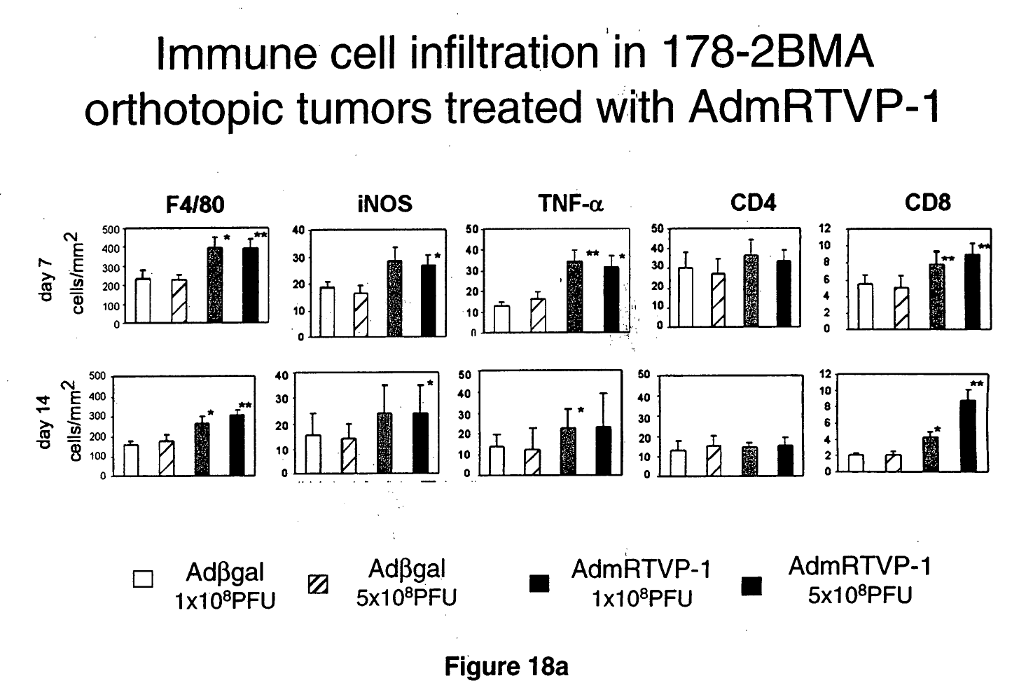

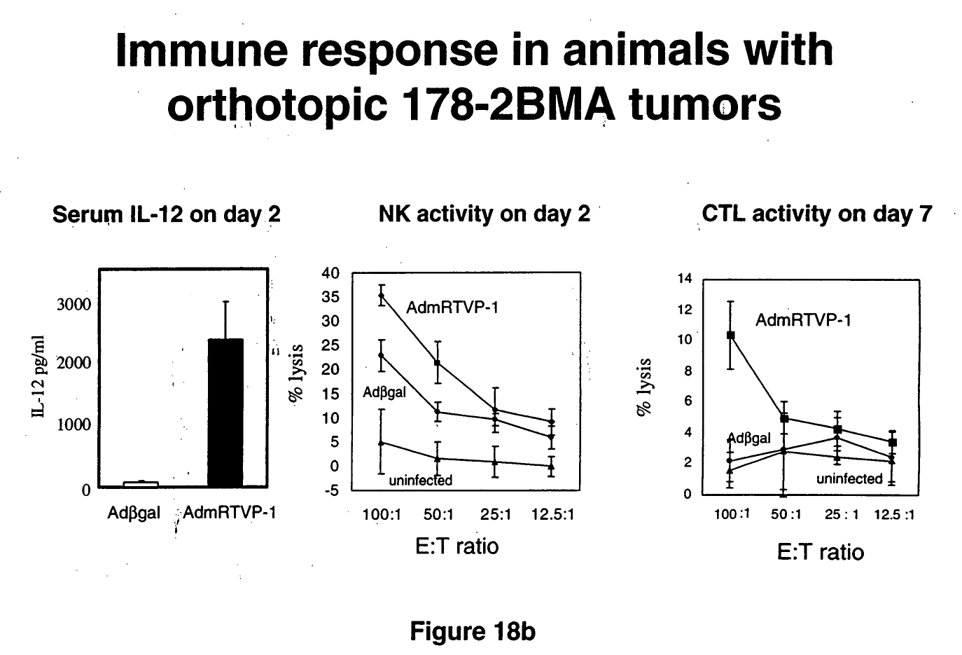

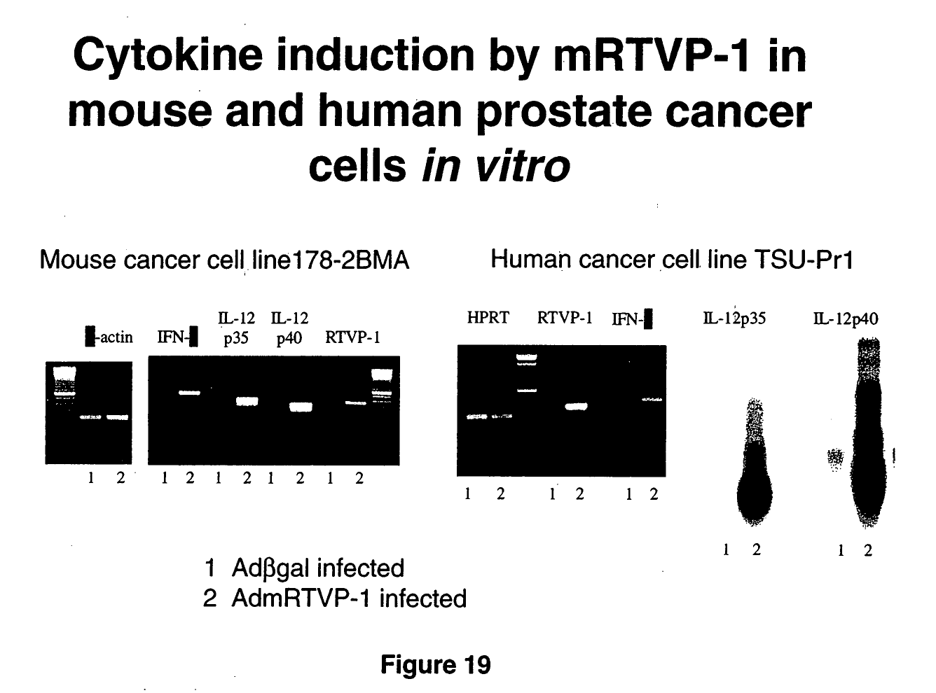

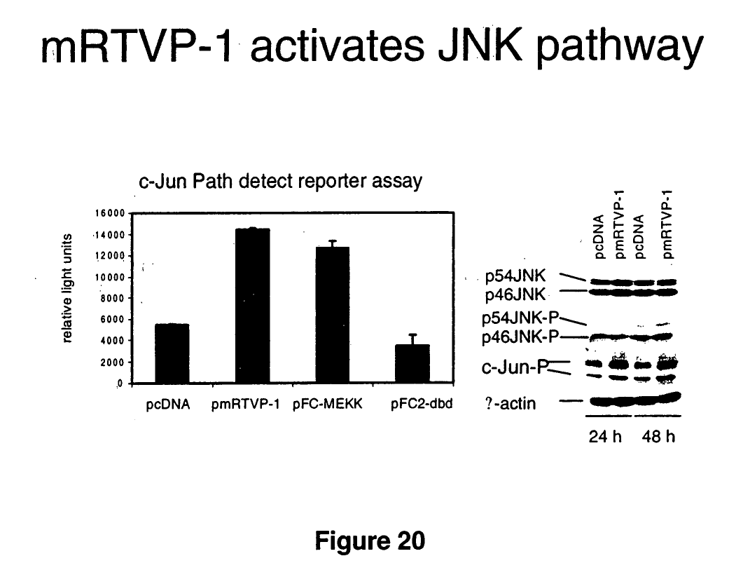

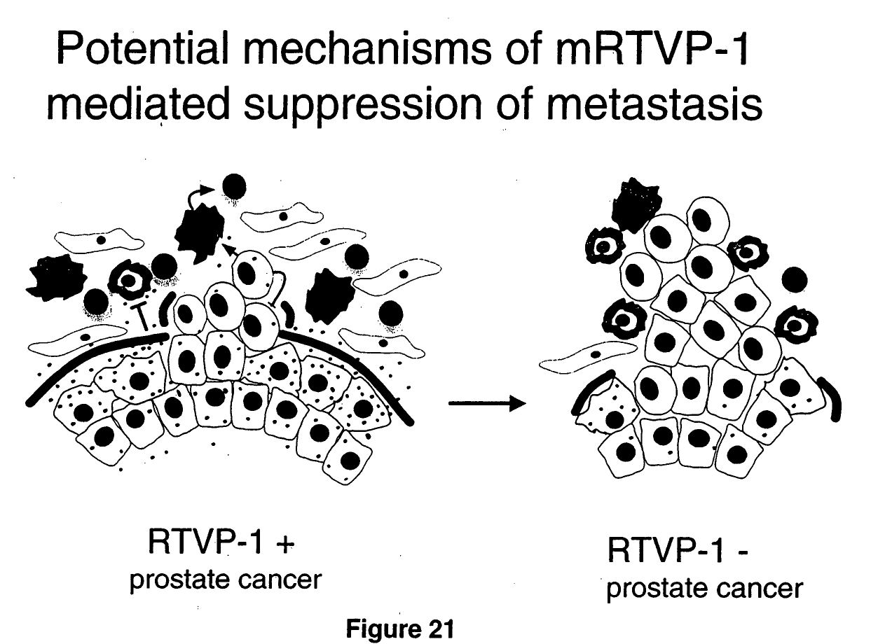

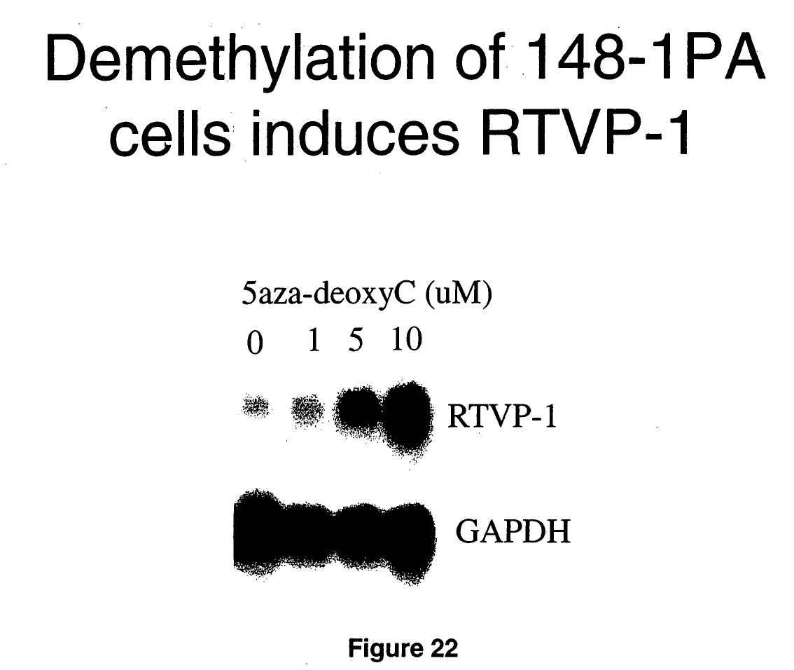

Experiments were also performed demonstrating a role for RTVP-1 in apoptosis of prostate cancer. A schematic is shown in FIG. 14, and the induction of apoptosis in 178-2BMA cells by AdmRTVP-1 in FIG. 15. Mice with orthotopic I 78-2BMA tumor tissue treated with AdmRTVP-1 showed increased survival (FIG. 16), as well as necrosis of the tumor tissues (FIG. 17). Immune cell infiltrates were seen (FIG. 18a) and an immune response for IL-12, NK activity and CTL activity (FIG. 18b). Cytokine induction was shown for both mouse and human prostate cancer in vitro (FIG. 19), and mRTVP-1 was shown to activate the JNK signal transduction pathway (FIG. 20). Potential mechanisms of mRTVP-1-mediated suppression of metastasis are shown in FIG. 21. Demethylation of 148-IPA cells with increasing amounts of 5aza-deoxyC was shown to induce RTVP-1 (FIG. 22).

### Example 7

### RTVP Effects on the Immune System and Immunological Responses

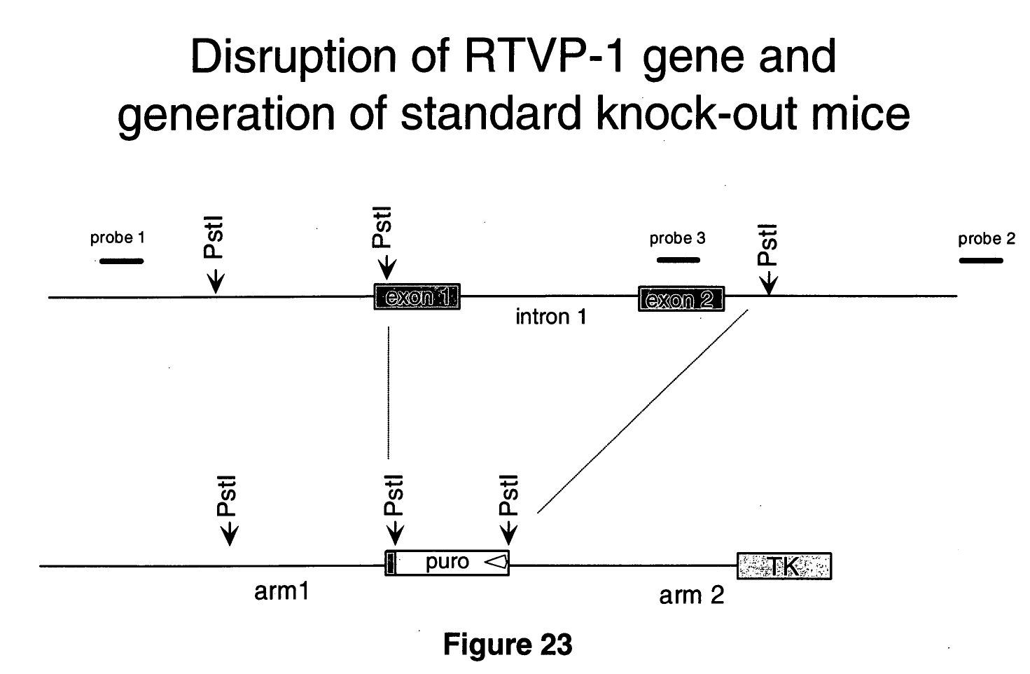

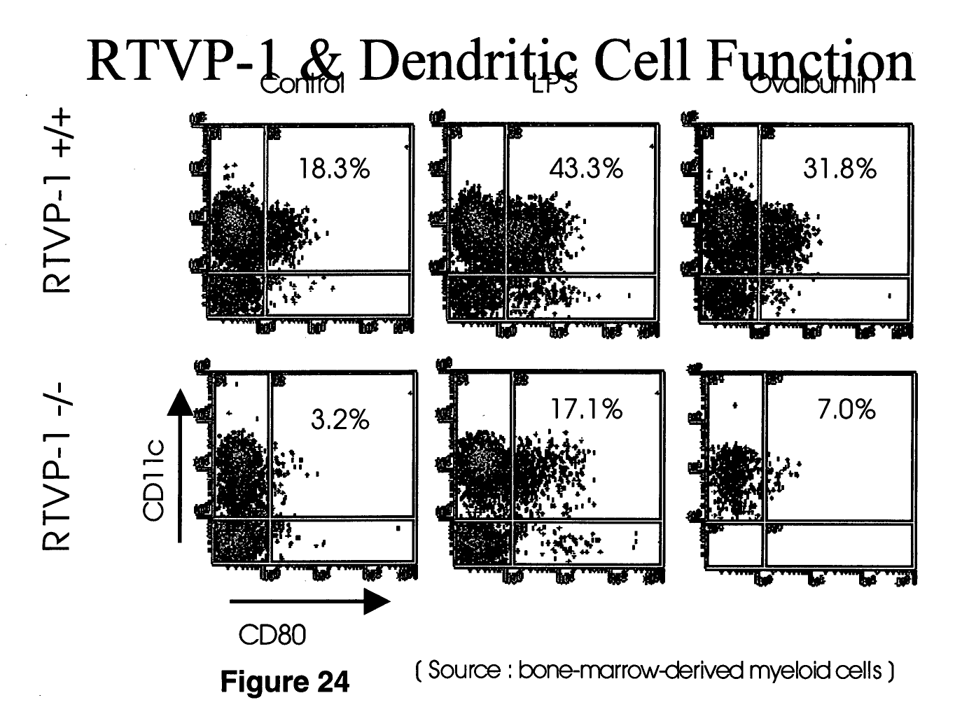

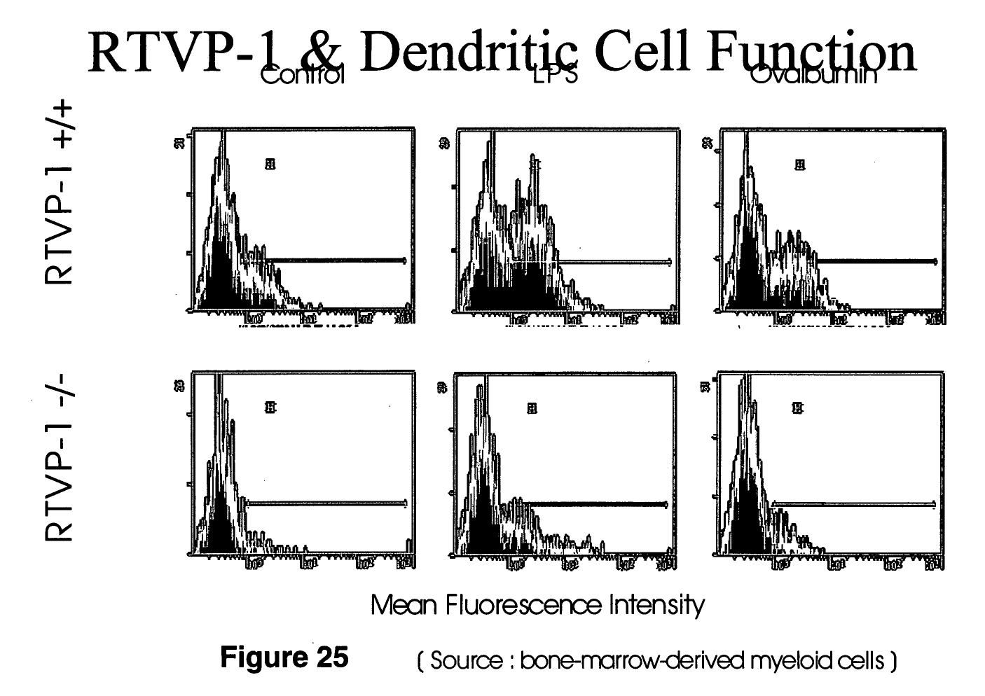

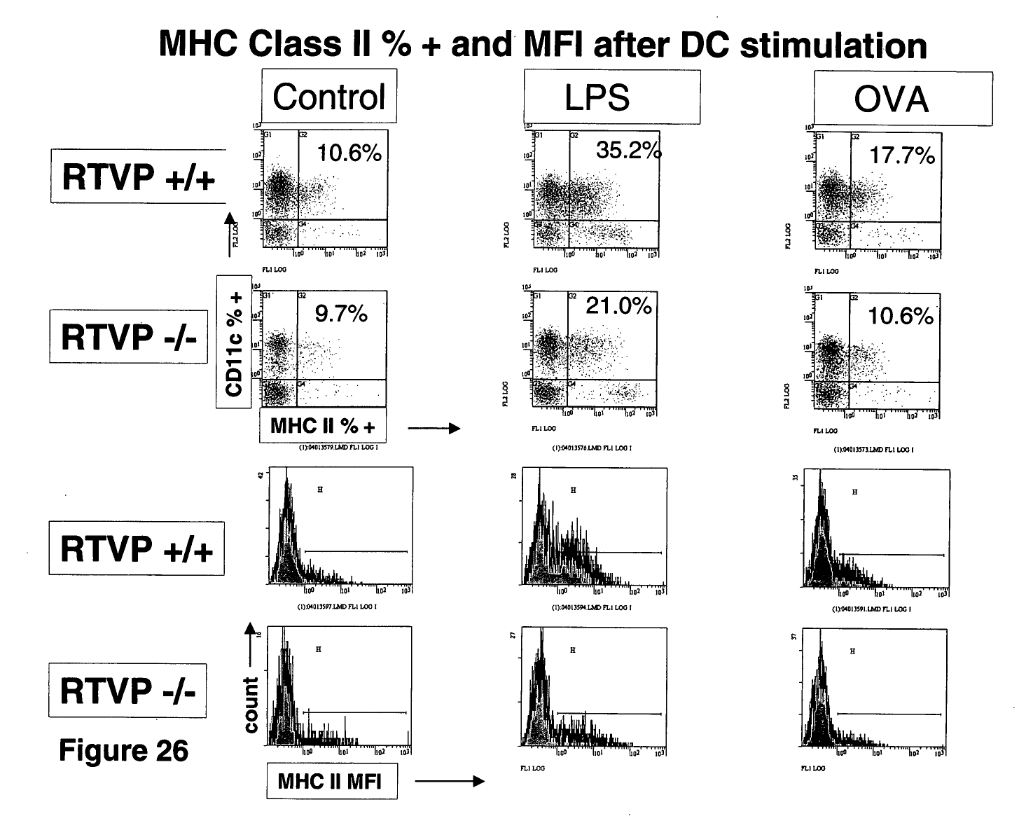

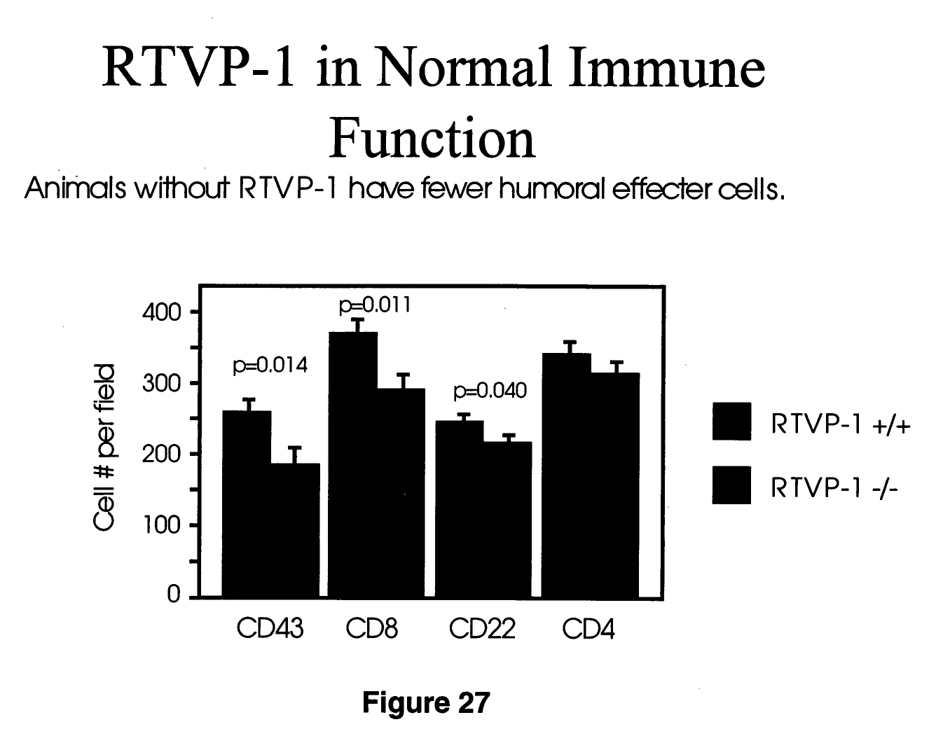

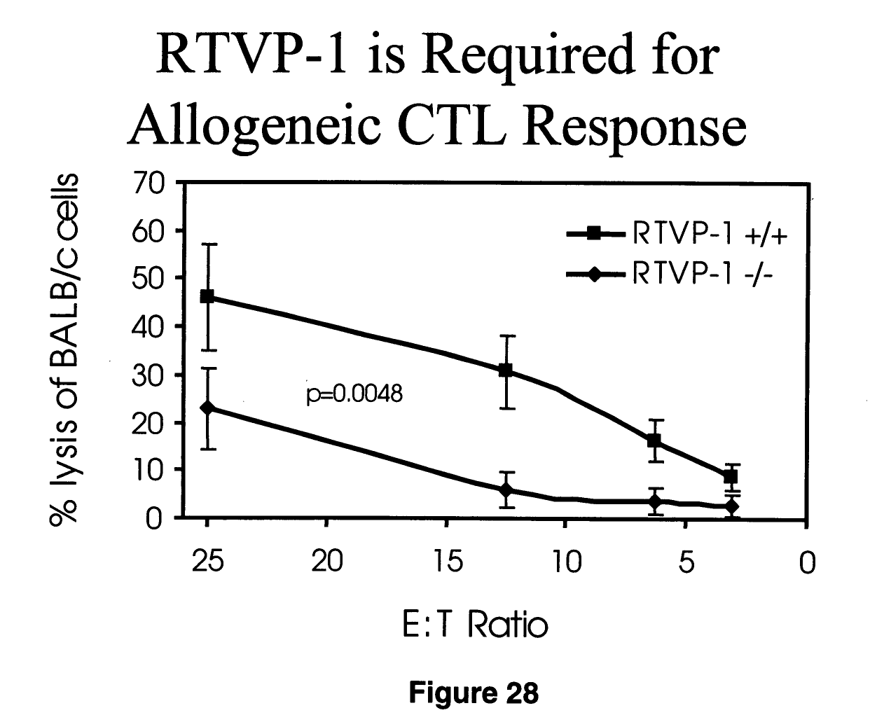

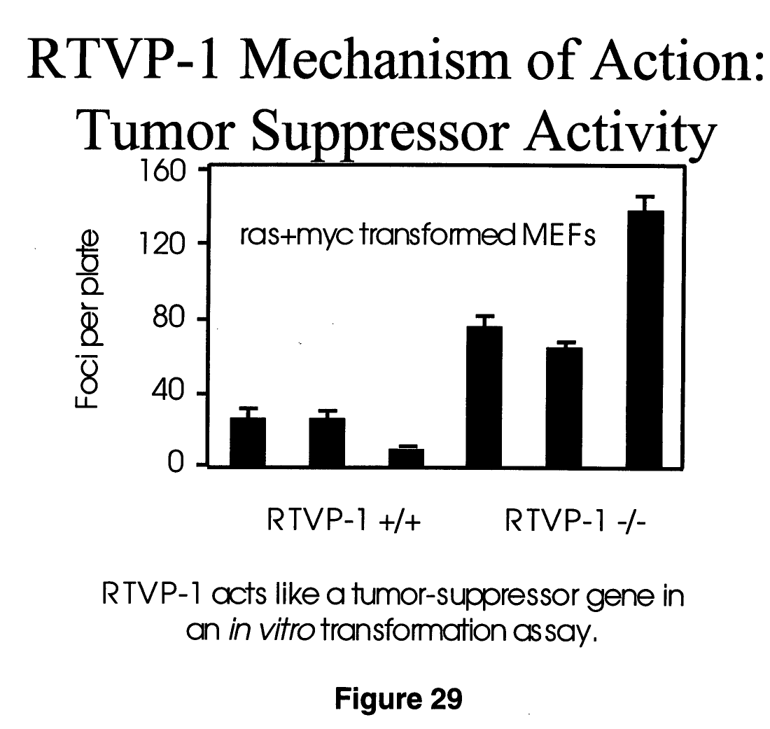

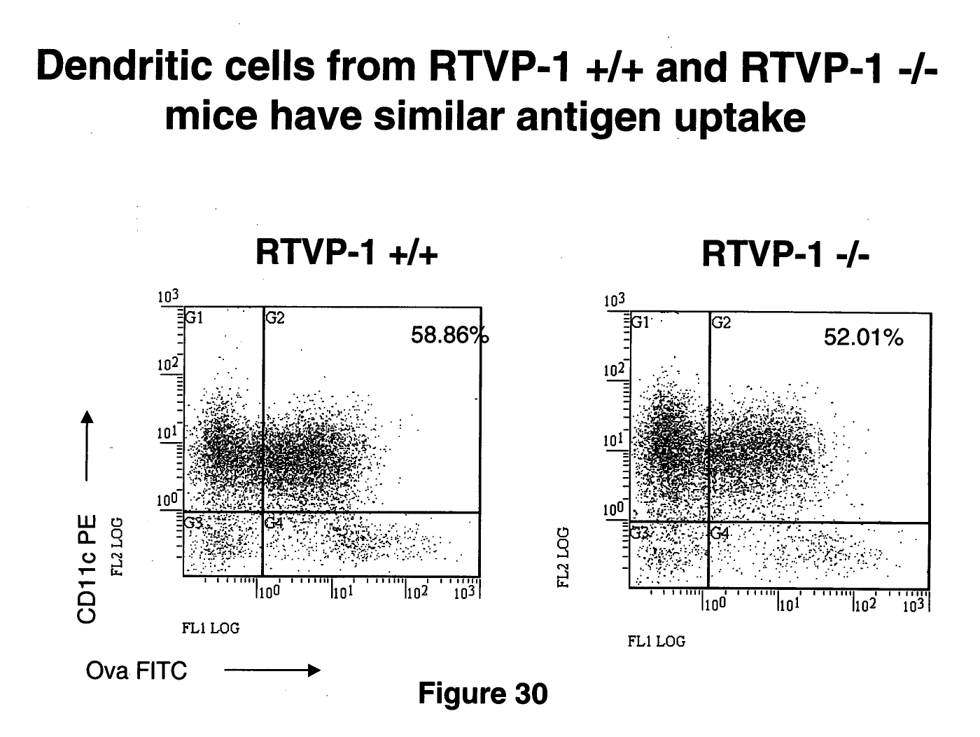

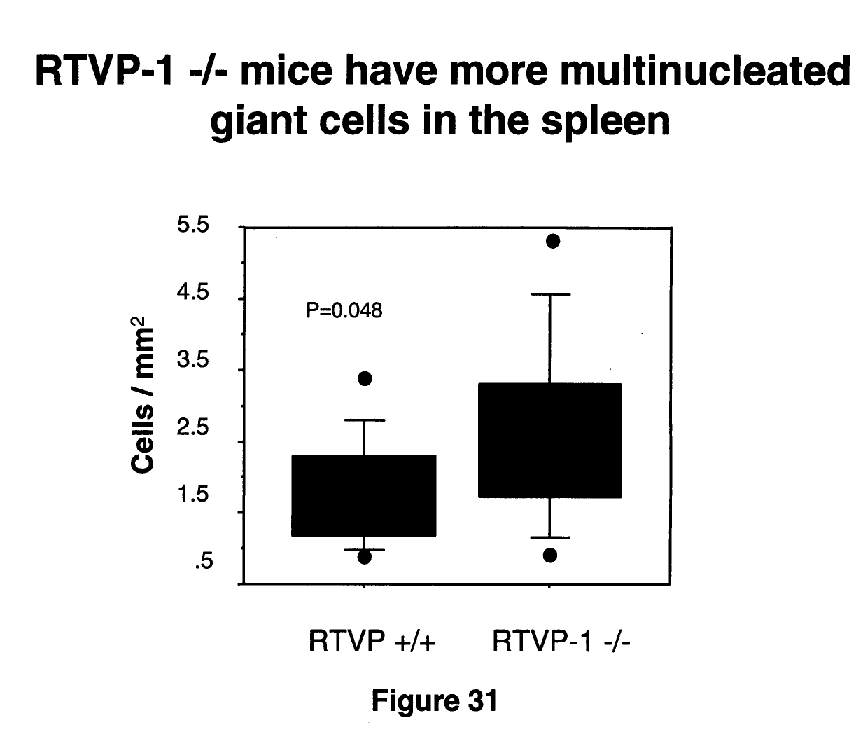

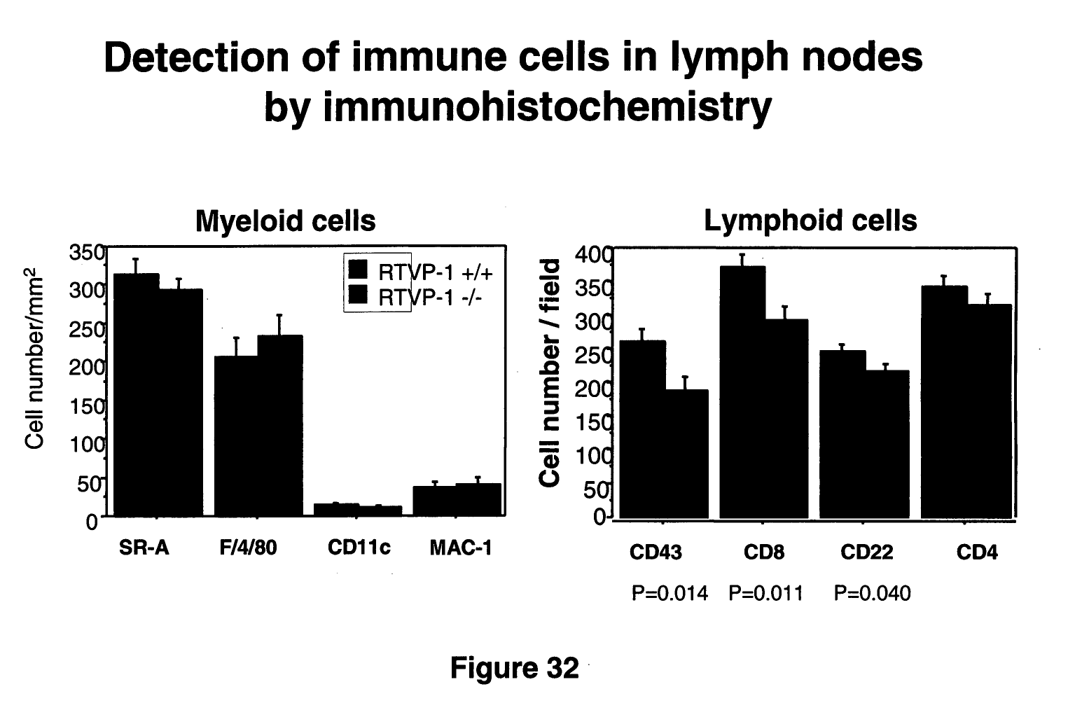

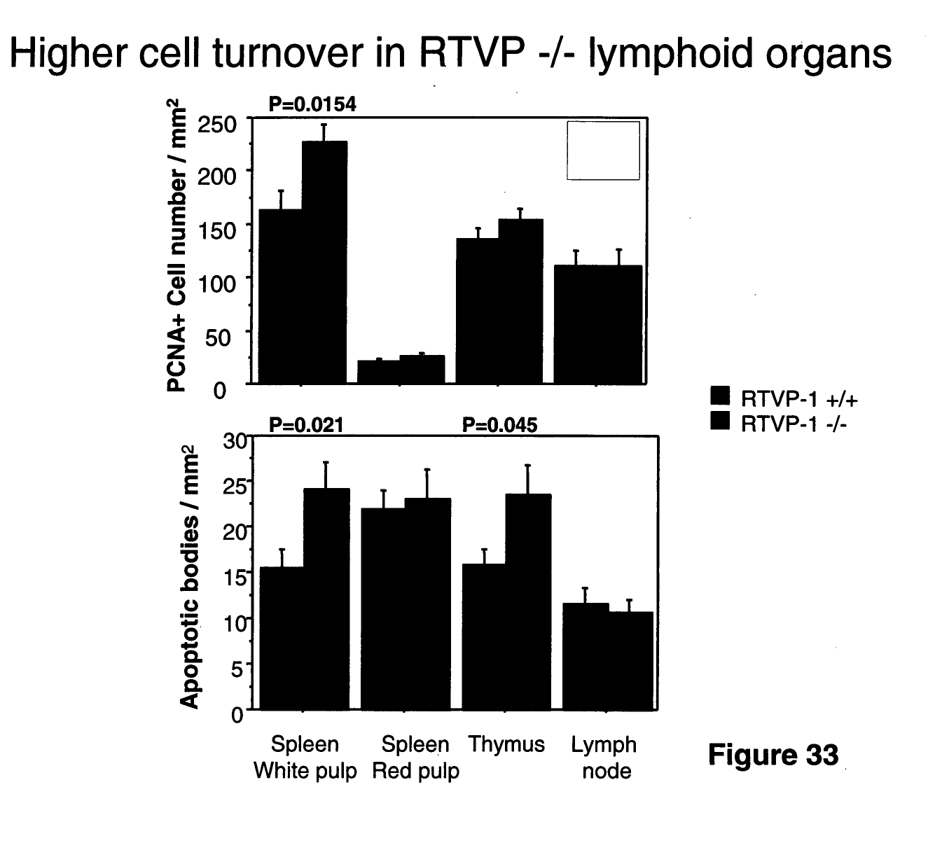

To further explore the underlying mechanisms responsible for mRTVP-1 mediated immunostimulatory activity, additional experiments were conducted. All experiments were performed as indicated or according to procedures which are well known to those of ordinary skill in the art. RTVP negative mice were created using standard knock-out technology (knock-out construct is shown in FIG. 23). Studies using homozygous RTVP negative mice in comparison to mice homozygous for the RTVP wild type indicated that loss of RTVP expression disrupted antigen presenting cell, specifically dendritic cell, activation and potentially antigen presentation. FIGS. 24 and 25 show that dendritic cells isolated and selected in culture from RTVP negative mice have a more restricted up-regulation of the activation marker CD80 upon stimulation of such cells by LPS or ovalbumin, as compared to similar cells isolated form homozygous wild type mice indicating that RTVP plays an important role in antigen presenting cell activation. In addition, FIG. 26 shows that loss of RTVP expression in the knock-out mice limited dendritic cell up-regulation of expression of MHC II by treatment with LPS or ovalbumin suggesting that RTVP may play a role in antigen presentation as well as antigen mediated activation. In situ analysis of humoral effecter cells in the lymph nodes of RTVP negative mice indicated that there are fewer lymphoid cells in these animals as compared to RTVP wild type mice (FIG. 27). Finally, studies of splenocytes from these RTVP negative mice demonstrated that CTL responses to allogenic cells were severely impaired (FIG. 28 showing the results of a standard CTL assay using allogenic splenocytes). These results suggest that the inhibition of RTVP expression could be useful in the treatment and prevention of autoimmune disorders and graft rejections by decreasing the immune systems ability to respond to antigenic challenge. On the other hand, these results also suggest that the administration of RTVP, or active fragments thereof, could increase or stimulate an immune response providing useful treatments for infectious diseases.

Other embodiments and uses of the invention will be apparent to those skilled in the art from consideration of the specification and practice of the invention disclosed herein. All references cited herein, including all publications, U.S. and foreign patents and patent applications including U.S. provisional application No. 60/209,989 entitled “P53 Regulated Gene Encoding p144-3 and p144-3 Protein,” filed Jun. 8, 2000, are specifically and entirely hereby incorporated herein by reference. It is intended that the specification and examples be considered exemplary only, with the true scope and spirit of the invention indicated by the following claims.

TABLE 1
Homology Comparison to Mouse RTVP-1 (p144-3 clone)

| | Percent Match |
|---|---|
| H. sapiens mRNA for RTVP protein | 53.7 |
| Human glioma pathogenesis-related protein | 60.5 |
| H. sapiens mRNA for cysteine-rich secretory protein | 49.6 |
| H. sapiens mRNA for cysteine-rich secretory protein | 49.7 |
| H. sapiens mRNA for cysteine-rich secretory protein | 47.5 |
| Acidic epididymal glycoprotein gene | 49.3 |
| Human mRNA for acidic epididymal glycoproteiri | 49.3 |
| Galago crassicaudatus encoding von Willebrand factor | 35.2 |
| Rat alpha2 urinary globulin gene | 23.5 |
| Human P450c21B gene coding for steroid | 34.6 |
| Human testes-specific protein (Tpx-1) | 47.1 |
| Mouse surfeit locus surfeit 3 gene | 35.3 |

TABLE 2
RTVP-1 mRNA expression in human prostate
specimens by in situ hybridization

| | RTVP-1 | | P53 staining^{‡} in RTVP-1 |
|---|---|---|---|
| | mRNA | | positive specimens % |
| | score^{†} | % (n) | (n) |
| Normal glandular cells | | | |
| | + | 0 (0/5) | |
| | ++ | 40 (2/5) | |
| | +++ | 60 (3/5) | |
| Primary cancer cells | | | |
| | + | 12 (2/16) | 50 (1/2) |
| | ++ | 69 (11/16) | 36 4/11) |
| | +++ | 19 3/16) | 0 (0/3) |
| Lymph node metastatic | | | |
| cancer cells* | | | |
| | + | 60 (3/5) | 67 (2/3) |
| | ++ | 40 (2/5) | 0 (0/2) |
| | +++ | 0 (0/5) | |

^{†}RTVP-1 mRNA in cells scored as: - no signal detected; + low; ++ moderate; or - +++ high level of fluorescence intensity.
^{‡}Presence or absence of clustered p53 staining (>15 cancer cell nuclei p53 + /300 × 400 μm² field)
*(P = .036, Mann-Whitney U test RTVP-1 mRNA score for lymph node metastatic cancer cells compared to primary cancer cells).

### REFERENCES

1. el-Deiry, W. S. Regulation of p53 downstream genes. Setnin Cancer Biol 8, 345-57 (1998).
2. Giaccia, A. J. & Kastan, M B. The complexity of p53 modulation: emerging patterns from divergent signals. Genes Dev 12, 2973-83 (1998).
3. Bums, T. F. & El-Deiry, W. S. The p53 pathway and apoptosis. J Cell Physiol 181, 231-9 (1999).
4. Vogelstein, B. & Kinzler, K. W. p53 function and dysfunction. Cell 70, 523-6 (1992).
5. Thompson, T. C., Timme, T. L. & Sehgal, I. Oncogenes, growth factors, and hormones in prostate cancer, in Hormones and growth factors in development and neoplasia. (eds. Dickson, R B. & Salomon, D. S.) 327-359 ( Wiley-Liss, Inc., New York, 1998).
6. Thompson, T. C. et. al. Loss of p53 function leads to metastasis in ras+myc-initiated mouse prostate cancer. Oncogene 10, 869-79 (1995).
7. Navone, N. M. et. al. p53 protein accumulation and gene mutation in the progression of -human prostate carcinoma. J Natl Cancer Inst 85, 1657-69 (1993).
8. Eastham, J. A. et. al. Association of p53 mutations with metastatic prostate cancer. Clin Cancer Res 1,1111-8(1995).
9. Mirchandani, D. et. al. Heterogeneity in intratumor distribution of p53 mutations in human prostate cancer. Am J Pathol 147, 92-101 (1995).
10. Yang, C. et. al. Clustered p53 immunostaining: a novel pattern associated with prostate cancer progression. Clin Cancer Res 2, 399-401 (1996).
11. Stapleton, A M. et. al. Primary human prostate cancer cells harboring p53 mutations are clonally expanded in metastases. Clin Cancer Res 3, 1389-97 (1997).
12. Quinn, D. I. et. al. Prognostic significance of p53 nuclear accumulation in localized prostate cancer treated with radical prostatectomy. Cancer Res 60, 1585-94 (2000).
13. Stapleton, A M. et. al. Assessment of the biologic markers p53, Ki-67, and apoptotic index as predictive indicators of prostate carcinoma recurrence after surgery. Cancer 82, 168-75 (1998).
14. Miyashita, T. & Reed, S. C. Tumor suppressor p53 is a direct transcriptional activator of the human bax gene. Cell 80, 293-9. (1995).
15. Sheikh, M S. et. al. p53-dependent and -independent regulation of the death receptor KILLER/DR5 gene expression in response to genotoxic stress and tumor necrosis factor alpha. Cancer Res 58, 1593-8 (1998).
16. Oda, K. et. al. p53AIP1, a potential mediator of p53-dependent apoptosis, and its regulation by Ser-46-phosphorylated p53. Cell 102, 849-62 (2000).
17. Zhu, J. & Chen, X. MCG1O, a novel p53 target gene that encodes a KH domain RNA-binding protein, is capable of inducing apoptosis and cell cycle arrest in G(2)-M. Mol Cell Biol 20, 5602-18 (2000).
18. Dameron, K. M., Volpert, O. V., Tainsky, M. A. & Bouck, N. Control of angiogenesis in fibroblasts by p53 regulation of thrombospondin-1. Science 265, 1582-4(1994).
19. Lopez-Ocejo, O. et. al. Oncogenes and tumor angiogenesis: the HPV-16 E6 oncoprotein activates the vascular endothelial growth factor (VEGF) gene promoter in a p53 independent manner. Oncogene 19, 4611-20(2000).
20. Zou, Z. et. al. p53 regulates the expression of the tumor suppressor gene maspin. J Biol Chem 275, 6051-4 (2000).
21. Zhang, M., Volpert, O., Shi, Y. H. & Bouck, N. Maspin is an angiogenesis inhibitor, Nat Med 6, 196-9 (2000).
22. Zhang, Y., Griffith, E. C., Sage, S., Jacks, T. & Liu, J. O. Cell cycle inhibition by the anti-angiogenic agent TNP-470 is mediated by p53 and p21WAF1/CIP1. Proc Natl Acad Sci USA 97, 6427-32 (2000).
23. Murphy, E. V., Zhang, Y., Zhu, W. & Biggs, J. The human glioma pathogenesis-related protein is structurally related to plant pathogenesis-related proteins and its gene is expressed specifically in brain tumors. Gene 159, 131-5. (1995).
24. Rich, T., Chen, P., Furman, F., Huynh, N. & Israel, M. A. RTVP-1, a novel human gene with sequence similarity to genes of diverse species, is expressed in tumor cell lines of glial but not neuronal origin. Gene 180, 125-30 (1996).
25. Gingras, M C. & Margolin, S. F. Differential expression of multiple unexpected genes during U937 cell and macrophage differentiation detected by suppressive subtractive hybridization. Exp Hematol 28, 65-76 (2000).
26. Cho, Y., Gorina, S., Jeffrey, P. D. & Pavletich, N. P. Crystal structure of a p53 tumor suppressor-DNA complex: understanding tumorigenic mutations. Science 265, 346-55 (1994).
27. Tokino, T. et. al. p53 tagged sites from human genomic DNA. Hum Mol Genet 3, 1537-42 (1994).
28. Vu, J. et. al. Identification and classification of p53-regulated genes. Proc Natl Acad Sci USA 96, 14517-22 (1999).
29. Shiraishi, K. et. al. Identification of fractalkine, a CX3C-type chemokine, as a direct target of p53. Cancer Res 60, 3722-6 (2000).
30. Bazan, J. F. et. al. A new class of membrane-bound chemokine with a CX3C motif. Nature 385, 640-4. (1997).
31. Pan, Y. et. al. Neurotactin, a membrane-anchored chemokine upregulated in brain inflammation. Nature 387, 611-7. (1997).
32. Sakr, W A. et. al. Allelic loss in locally metastatic, multisampled prostate cancer. Cancer Res 54, 3273-7 (1994).
33. Qian, J. et. al. Chromosomal anomalies in prostatic intraepithelial neoplasia and carcinoma detected by fluorescence in situ hybridization. Cancer Res 55, 5408-14 (1995).
34. Ren, C., Yang, G., Timme, T. L., Wheeler, T. M. & Thompson, T. C. Reduced lysyl oxidase messenger RNA levels in experimental and human prostate cancer. Cancer Res 58, 1285-90 (1998).
35. Yang, G. et. al. Elevated expression of caveolin is associated with prostate and breast cancer. Clin Cancer Res 4,1873-80(1998).
36. Nasu, Y. et. al. Suppression of caveolin expression induces androgen sensitivity in metastatic androgen-insensitive mouse prostate cancer cells. Nat Med 4, 1062-4 (1998).
37. Baker, S. J., Markowitz, S., Fearon, E. R., Willson, J. K. & Vogelstein, B. Suppression of human colorectal carcinoma cell growth by wild-type p53. Science 249, 912-5. (1990).
38. Timme, T. L. et. al. Caveolin-1 is regulated by c-myc and suppresses c-myc-induced apoptosis. Oncogene 19, 3256-65 (2000).
39. Patel, S. D. et. al. The p53-independent tumoricidal activity of an adenoviral vector encoding a p27-p16 fusion tumor suppressor gene. Mol Ther 2, 161-9. (2000).
40. Hall, S. J. et. al. Cooperative therapeutic effects of androgen ablation and adenovirusmediated herpes simplex virus thymidine kinase gene and ganciclovir therapy in experimental prostate cancer. Cancer Gene Ther 6, 54-63 (1999).
41. Nasu, Y. et. al. Adenovirus-mediated interleukin-12 gene therapy for prostate cancer: suppression of orthotopic tumor growth and pre-established lung metastases in an orthotopic model. Gene Ther 6, 338-49 (1999).
42. Eastham, J. A. et. al. In vivo gene therapy with p53 or p21 adenovirus for prostate cancer, Cancer Res 55, 5151-5. (1995).
43. Baley, P. A., Yoshida, K., Qian, W., Sehgal, I. & Thompson, T. C. Progression to androgen insensitivity in a novel in vitro mouse model for prostate cancer. J Steroid Biochem Mol Biol 52, 403-13 (1995).
44. Osoegawa, K. et. al. Bacterial artificial chromosome libraries for mouse sequencing and functional analysis. Genome Res 10, 116-28. (2000).

1. A method for treating a patient comprising administering to the patient a therapeutically effective amount of a composition comprising at least an active portion of an RTVP protein having homology to a mammalian testes-specific protein, a plant pathogenesis-related protein, a vespid venom allergan, or a combination thereof, wherein the active portion has immune system modulating activity.

2. The method of claim 1, wherein the immune system modulating activity is a stimulation of the immune system.

3. The method of claim 2, wherein the patient is being treated for an infectious disease.

4. The method of claim 1, wherein the immune system modulating activity is a suppression of the immune system.

5. The method of claim 4, wherein the patient is being treated for an autoimmune disorder.

6. The method of claim 5, wherein the autoimmune disorder is selected from the group consisting of allergic reactions, Crohn's disease, Grave's disease, diabetes, arthritis, lupus, multiple sclerosis, myasthenia gravis, cirrhosis, graft versus host disease, transplant rejections, and ulcerative colitis.

7. The method of claim 1, wherein the composition further comprises a pharmaceutically acceptable carrier selected from the group consisting of water, oils, alcohols, salts, fatty acids, saccharides, polysaccharides and combinations thereof.

8. A method for treating a patient comprising administering to the patient a therapeutically effective amount of a composition comprising an isolated RTVP receptor protein or portion thereof which binds to an RTVP polypeptide, wherein the RTVP receptor has immune system modulating activity.

9. An antibody which binds to an RTVP polypeptide and has immune system modulating activity.

10. The antibody of claim 9, wherein the antibody is a monoclonal antibody.

11. A method for treating a patient comprising administering to the patient a therapeutically effective amount of the antibody of claim 9.

12. The method of claim 11, wherein the immune system modulating activity is a stimulation of the immune system.

13. The method of claim 12, wherein the patient is being treated for an infectious disease.

14. The method of claim 11, wherein the immune system modulating activity is a suppression of the immune system.

15. The method of claim 14, wherein the patient is being treated for an autoimmune disorder.

16. The method of claim 15, wherein the autoimmune disorder is selected from the group consisting of allergic reactions, Crohn's disease, Grave's disease, diabetes, arthritis, lupus, multiple sclerosis, myasthenia gravis, cirrhosis, graft versus host disease, transplant rejections, and ulcerative colitis.

17. An isolated nucleic acid comprising all or a portion of SEQ ID NO: 1.

18. The nucleic acid of claim 17, wherein the nucleic acid encodes a polypeptide having an immune system modulating activity.

19. The nucleic acid of claim 18, wherein the characteristic portion is selected from the group consisting of an open reading frame of the encoded polypeptide, a conserved domain of the nucleic acid, a conserved domain of the encoded protein, an intron, an exon and combinations thereof.

20. The nucleic acid of claim 17, wherein the immune system modulating activity is an activation of the immune system.

21. The nucleic acid of claim 20, wherein the activation of the immune system comprises activation of antigen presenting cells.

22. The nucleic acid of claim 21, wherein the activation of the immune system comprises the induction of CD80 expression by the antigen present cells.

23. The nucleic acid of claim 17, wherein the immune system modulating activity is a suppression of the immune system.

24. An isolated polypeptide which comprises SEQ ID NO. 2, or an active portion thereof, wherein said polypeptide has an immune system modulating activity.

25. The polypeptide of claim 24, wherein the amino acid sequence is selected from the group consisting of an exonic region of the polypeptide, a conserved domain of the polypeptide, a functional domain of the polypeptide, an antigenic domain of the polypeptide, a p53 binding site of the polypeptide, and combinations thereof.

26. The polypeptide of claim 24, wherein the immune system modulating activity is an activation of the immune system.

27. The polypeptide of claim 26, wherein the activation of the immune system comprises activation of antigen presenting cells.

28. The polypeptide of claim 27, wherein the activation of the immune system comprises the induction of CD80 expression by the antigen present cells.

29. The polypeptide of claim 24, wherein the immune system modulating activity is a suppression of the immune system.
